(19) United States
(12) Patent Application Publication
(10) Pub. No.: US 2011/0212504 A1
(43) Pub. Date: Sep. 1, 2011

(54) RECOMBINANT EXPRESSION OF CARBOXYLESTERASES

(75) Inventor: Zhongbin Liu, Shanghai (CN)

(73) Assignee: TONGJI UNIVERSITY, Shanghai (CN)

(21) Appl. No.: 13/127,652

(22) PCT Filed: Jun. 2, 2010
(86) PCT No.: PCT/CN2010/073449

§ 371 Date: May 4, 2011

(30) Foreign Application Priority Data
Aug. 31, 2009 (CN) 200910166839.1

(51) Int. Cl. C12N 9/18; C12N 1/15; C12N 1/19

(52) U.S. Cl. 435/197; 435/256.1; 435/255.5

(57) ABSTRACT

The present application provides a method of producing a carboxylesterase or its variant in eukaryotic cells. The present application also provides an expression vector for high level carboxylesterase expression, a eukaryotic cell comprising the expression vector, and uses thereof. The present application also provides a composition comprising a carboxylesterase or its variant produced by a method described in the present application and uses of the composition.

### CROSS-REFERENCE TO RELATED APPLICATIONS

For purposes of the USPTO extra-statutory requirements, the present application claims benefit of priority of Chinese Patent Application No. 200910166839.1, entitled Recombinant Expression of Carboxylesterase, naming Zhongbin Liu as inventor, filed 31, Aug., 2009, which was filed within the twelve months preceding the filing date of the present application, or is an application of which a currently co-pending application is entitled to the benefit of the filing date.

All subject matter of the listed applications and of any and all parent, grandparent, great-grandparent, etc. applications of the Related Applications is incorporated herein by reference to the extent such subject matter is not inconsistent herewith.

### BACKGROUND

A carboxylesterase can hydrolyze a carboxylic ester to produce a carboxylate and an alcohol. Carboxylesterases belong to the superfamily of hydrolases. Carboxylesterases have been identified in various species from prokaryotic cells to eukaryotic cells.

### SUMMARY

In one aspect, the present disclosure provides a method for producing a protein, comprising culturing a eukaryotic cell engineered to express a gene encoding for a carboxylesterase or its variant under conditions suitable for expression of the carboxylesterase or its variant.

In another aspect, the present disclosure provides a method for producing a protein, comprising culturing a eukaryotic cell engineered to express a gene encoding for a microbial carboxylesterase or its variant under conditions suitable for expression of the microbial carboxylesterase or its variant.

In another aspect, the present disclosure provides a method for producing a protein, comprising culturing a filamentous fungal cell engineered to express a gene encoding for a carboxylesterase or its variant under conditions suitable for expression of the carboxylesterase or its variant.

In another aspect, the present disclosure provides an expression vector, comprising a gene encoding for a carboxylesterase or its variant; and a regulatory sequence capable of promoting expression of the carboxylesterase or its variant in a eukaryotic cell, wherein the regulatory sequence is operably linked to the gene.

In another aspect, the present disclosure provides a eukaryotic cell comprising an expression vector containing a gene encoding for a carboxylesterase or its variant, and a regulatory sequence capable of promoting expression of the carboxylesterase or its variant in the eukaryotic cell, wherein the regulatory sequence is operably linked to the gene.

In another aspect, the present disclosure provides a composition comprising a eukaryotic cell and a carboxylesterase or its variant expressed by the eukaryotic cell.

In another aspect, the present disclosure provides a composition comprising a filamentous fungal cell and a carboxylesterase or its variant expressed by the filamentous fungal cell.

In another aspect, the present disclosure provides a composition comprising an isolated carboxylesterase or its variant produced by a method of the present disclosure.

In another aspect, the present disclosure provides methods of using the compositions of the present disclosure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 shows a schematic map of plasmid pIGF.

FIG. 2 shows a schematic map of plasmid pYG1.2.

FIG. 3 shows the gel electrophoresis results of the culture media from Aspergillus niger M54 transformed with pYG1.2-CarE-his (L2), Aspergillus niger M54 transformed with pYG1.2 (L3), and non-transformed Aspergillus niger M54 (L4). The protein molecular weight markers are shown in L1. The band around 29.0 KD shown in L2 is the band for the carboxylesterase.

FIG. 4 shows the Western blot results of the carboxylesterase expressed from Pichia pastoris GS115 transformed with pPIC9K-CarE-His (L4) and from Aspergillus niger M54 transformed with pYG1.2-CarE-His (L5). Expression of carboxylesterase is not detected in the negative controls: Pichia pastoris GS115 transformed with pPIC9K (L3), non-transformed Aspergillus niger M54 (L6), Aspergillus niger M54 transformed with pYG1.2 (L 7). The positive control (a His-tag containing protein) is shown in L1 and protein markers are shown in L2.

FIG. 5 shows the gel electrophoresis results of carboxylesterase expressed from Pichia pastoris GS115 transformed with pPIC9K-CarE-His sampled after cultivation for 24 h (L3), 48 h (L4) and 72 h (L5). Protein markers are shown in L1 and culture medium of Pichia pastoris GS115 transformed with pPIC9K is shown in L2.

FIG. 6 shows the enzymatic activities of carboxylesterases isolated from culture media at different time points of incubation.

FIG. 7 shows the relative enzymatic activities of recombinant carboxylesterase measured at different pH values.

FIG. 8 shows the relative enzymatic activities of recombinant carboxylesterase measured at 37° C. after the carboxylesterase has been treated at different temperatures for 10 or 30 minutes.

FIG. 9 shows the relative enzymatic activities of recombinant carboxylesterase measured at different temperatures.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The present disclosure relates to recombinant methods for producing carboxylesterases and variants thereof, expression vectors and host cells useful for recombinantly producing carboxylesterases and variants thereof. The present disclosure also relates to compositions comprising the recombinantly produced carboxylesterases and variants thereof and methods of using the compositions.

In one aspect, the present disclosure provides a method for producing a protein comprising culturing a eukaryotic cell engineered to express a gene encoding for a carboxylesterase or its variant under conditions suitable for expression of the carboxylesterase or its variant.

In another aspect, the present disclosure provides a method for producing a protein comprising culturing a filamentous fungal cell engineered to express a gene encoding for a carboxylesterase or its variant under conditions suitable for expression of the carboxylesterase or its variant.

In another aspect, the present disclosure provides a method for producing a protein comprising culturing a eukaryotic cell engineered to express a gene encoding for a microbial carboxylesterase or its variant under conditions suitable for expression of the carboxylesterase or its variant.

### Eukaryotic Cells

Eukaryotic cells are cells that are organized into complex structures enclosed within membranes, which include, inter alia, a membrane-bound nucleus containing genetic materials. The eukaryotic cells of this disclosure include, without limitation, fungal cells, protist cells, animal cells and plant cells.

Fungal cells may include, without limitation, yeast cells and filamentous fungal cells.

Yeast cells of the present disclosure may belong to the division of Ascomycota and Basidiomycota by their phylogenetic characteristics. Illustrative examples of yeast cells include, without limitation, Pichia species such as Pichia angusta, Pichia pastoris, Pichia anomala, Pichia stipitis, Pichia methanolica, and Pichia guilliermondii; Hansenula species such as Hansenula anomala, Hansenula polymorpha, Hansenula wingei, Hansenula jadinii and Hansenula saturnus; Saccharomyces species such as Saccharomyces cerevisiae, Saccharomyces bayanus, Saccharomyces boulardii; Candida species such as Candida albicans, Candida methylica, Candida boidinii, Candida tropicalis, Candida wickerhamii, Candida maltosa, and Candida glabrata, Torulopsis glabrata; and also Kluyveromyces species, and Schizosaccharomyces species.

In certain embodiments, the yeast cell is one or more of Pichia pastoris, Hansenula polymorpha, Saccharomyces cerevisiae, or Torulopsis glabrata. In certain embodiments, the yeast cell is Pichia pastoris. In certain embodiments, the yeast cell is one or more of Pichia pastoris strain GS115 cell, Pichia pastoris strain KM71 cell or Pichia pastoris strain MC100-3 cell. In certain embodiments, the yeast cell is a Hansenula polymorpha strain ATCC34438 cell.

Filamentous fungi may include without limitation any species of microscopic fungi that grow in the form of multi-cellular filaments. In certain embodiments, the filamentous fungal cells include, but are not limited to, the various species of Acremonium, Aspergillus, Fusarium, Humicola, Mucor, Myceliophthora, Neurospora, Penicillium, Thielavia, Tolypocladium, and Trichoderma.

In certain embodiments, the filamentous fungal cell is an Aspergillus awamori, Aspergillus foetidus, Aspergillus japonicus, Aspergillus nidulans, Aspergillus niger or Aspergillus oryzae cell. In an illustrative embodiment, the filamentous fungal cell is an Aspergillus niger ATCC 12049 strain cell. In another illustrative embodiment, the filamentous fungal cell is an Aspergillus oryzae RIB40 strain cell.

Protist cells may include, without limitation, protozoa cells and algae cells.

Animal cells may include, without limitation, mammalian cells, avian cells, amphibian cells, and insect cells. Illustrative examples of animal cells include pig liver cells, human embryonic kidney 293 (HEK293) cells, Chinese hamster ovary cells (CHO), zebrafish PAC2 cells, Xenopus A6 kidney epithelial cells, caenorhabditis elegans cells, and drosophila cells.

Plant cells may include, without limitation, parenchyma cells, collenchyma cells, and sclerenchyma cells. Illustrative examples of plant cells are Tobacco BY-2 cells, Datura innoxia cell line, and SB-1 cell line.

In certain embodiments, the eukaryotic cells in the present disclosure may carry one or more mutations that cause phenotype changes from the wild type strains. Mutations in the eukaryotic cells may occur naturally or non-naturally. Naturally occurring mutations may form spontaneously in the course of evolution. Non-naturally occurring mutations may be artificially generated using methods known in the art. In an illustrative example, mutations may be generated by exposing cells to physical mutagens such as UV irradiation or chemical mutagens such as hydroxylamine and ethidium bromide (see, for example, Hopwood, The Isolation of Mutants in Methods in Microbiology (J. R. Norris and D. W. Ribbons, eds.) 1970, 363-433, Academic Press, New York). In another illustrative example, mutations may be generated by gene deletion techniques such as homologous recombination to disrupt the expression of one or more target genes (see, for example, Alberts et al, Chapter 5: DNA Replication, Repair, and Recombination, Molecular biology of the cell, 2002, 845, Garland Science. New York). In another illustrative example, mutations may be made by gene modification techniques such as polymerase chain reaction (PCR) (see, for example, Botstein et al, Strategies and applications of in vitro mutagenesis, Science 1985, vol 229, No. 4719, 1193-1201; Lo et al., Specific amino acid substitutions in bacterioopsin: Replacement of a restriction fragment in the structural gene by synthetic DNA fragments containing altered codons, Proc. Natl. Acad. Sci. USA 1985, vol 81, No. 8, 2285-2289; Youngman et al., Genetic transposition and insertional mutagenesis in Bacillus subtilis with Streptococcus faecalis transposon Tn917, Proc. Natl. Acad. Sci. USA 1983, vol 80, No. 8, 2305-2309).

In certain embodiments, the eukaryotic cells in the present disclosure may carry one or more mutations that render them unable to synthesize an essential substance required for cell growth. Mutations may occur in genes involved in the synthesis and/or metabolism of amino acids, nucleotides, sugars, fatty acids, vitamins and other essential substances.

In certain embodiments, the eukaryotic cells may be mutant yeast cells carrying mutations in ura, trp, ade and leu genes which are involved in the synthesis of uridine, tryptophan, adenosine, and leucine, respectively (Agaphonov et al., Isolation and characterization of the LEU2 gene of Hansenula polymorpha, Yeast 1994, vol 10, 509-513; Bogdanova et al., Plasmid eorganization during integrative transformation in Hansenula polymorpha, Yeast 1995, vol 11, 343-353; Merckelbach et al., Cloning and sequencing of the ura3 locus of the methylotrophic yeast Hansenula polymorpha and its use for the generation of a deletion by gene replacement, Appl. Microbiol. Biotechnol. 1993, vol 40, 361-364).

In certain embodiments, the eukaryotic cells may be mutant filamentous fungal cells, e.g. Aspergillus niger strains, deficient in pyrG gene function, which are unable to synthesize uridine and thus cannot grow on uridine-free culture medium (Liu, et al, Construction of pyrG auxotrophic Aspergillus niger strain, Journal of microbiology 2001, vol 21, No. 3, 15-16). In an illustrative embodiment, the eukaryotic cell is Aspergillus niger M54, which has been deposited with the China Center for Type Culture Collection (CCTCC), Wuhan University, Wuhan, China, on Jun. 14, 2009, and assigned the Accession No. CCTCC M 209121, under the terms and conditions of the Budapest Treaty on the International Recognition of the Deposit of Microorganisms for the Purpose of Patent Procedure (the Budapest Treaty).

In another illustrative embodiment, the eukaryotic cells are auxotrophic Aspergillus oryzae mutant strains, for example, Aspergillus oryzae M-2-3 deficient in argB gene, which are unable to synthesize arginine and thus cannot grow on arginine-free culture medium (Gomi et al, Integrative transformation of Aspergillus oryzae with a plasmid containing the Aspergillus nidulans argB gene. Agric Biol. Chem. 1987, vol 51, 2549-2555), and Aspergillus oryzae deficient in niaD gene, which are deficient in nitrate reductase and thus are unable to grow on the medium with nitrate as sole source of nitrogen (Unkles et al, The development of a homologous transformation system for Aspergillus oryzae based on the nitrate assimilation pathway: A convenient and general selection system for filamentous fungal transformation, Molecular and General Genetics 1989, vol 218, No. 1, 99-104).

### Carboxylesterases and Variants Thereof

The term “carboxylesterase” as used herein refers to an enzymatic polypeptide that is capable of hydrolyzing a carboxyl ester into a carboxylate and an alcohol. A carboxylesterase may be a wild type carboxylesterase or any variant thereof. A variant of a wild type carboxylesterase differs from the wild type carboxylesterase in the amino acid sequence and/or modification of the amino acids but retains the capability to hydrolyze a carboxyl ester into a carboxylate and an alcohol. The variant may have one or more amino acid substitutions, additions, deletions, insertions, truncations, modifications (e.g. phosphorylation, glycosylation, labeling, etc.), or any combination thereof, of the wild type carboxylesterase. The variant may include naturally occurring variants of the wild type carboxylesterase and artificial polypeptide sequences such as those obtained by chemical synthesis or recombinant methods. The variant may include fragments, mutants, hybrids, analogs and derivatives of wild type carboxylesterases. The variants may contain non-naturally occurring amino acid residues.

Carboxylesterases have been identified and isolated from a wide variety of species, including, without limitation, animals, insects, plants, and microbes. The nucleotide sequences and amino acid sequences of carboxylesterases from many species have been identified.

In one embodiment, the carboxylesterase is derived from microbes. The term “microbe” refers to any living organism other than humans, animals and plants. Microbes may include, without limitation, prokaryotes such as bacteria, protozoa, fungi, protists and archaea. Illustrative examples of microbes are Escherichia coli, Geobacillus stearothermophilus, Bacillus cereus, Candida rugosa, Plasmodium falciparum, Pyrococcus furiosus, Salmonella enterica, and Aspergillus fumigatus.

Carboxylesterases have been isolated from many microbes and their corresponding nucleotide sequences and amino acid sequences have been obtained. Table 1 lists illustrative examples of microbial carboxylesterases and their nucleotide and polypeptide sequences identified by GenBank Accession Numbers.

TABLE 1
Illustrative examples of carboxylesterases of different microbes.

| | GenBank Amino Acid | GenBank Nucleotide |
|---|---|---|
| Species | Sequence Accession No. | Sequence Accession No. |
| Geobacillus | BAD77330 | BA000043, Region: |
| kaustophilus | (SEQ ID NO: 1) | 3067043 . . . 3067783 |
| | | (SEQ ID NO: 2) |
| Geobacillus | AAG53982 | AF327065 |
| thermoleovorans | (SEQ ID NO: 3) | (SEQ ID NO: 4) |
| Salmonella enterica | YP_002245400 | NC_011294, Region: |
| | (SEQ ID NO: 5) | 3548658 . . . 3549428 |
| | | (SEQ ID NO: 6) |
| Aspergillus | XP_755184 | XM_750091 |
| fumigatus | (SEQ ID NO: 7) | (SEQ ID NO: 8) |

In one embodiment, the carboxylesterase is derived from bacteria. Illustrative examples of bacteria are, Escherichia coli, Geobacillus stearothermophilus, Geobacillus kaustophilus, Sulfolobus solfataricus, and Bacillus thermoleovorans. In another embodiment, the carboxylesterase is derived from thermophilic bacteria. Illustrative examples of thermophilic bacteria are Geobacillus stearothermophilus, Geobacillus kaustophilus, Sulfolobus solfataricus, and Bacillus thermoleovorans. In another embodiment, the carboxylesterase is derived from Geobacillus stearothermophilus. Four carboxylesterases have been identified from Geobacillus stearothermophilus. The amino acid sequences and nucleotide sequences of the four carboxylesterases are set forth in SEQ ID NOs: 9-16 as shown in Table 2 below.

TABLE 2
Illustrative examples of carboxylesterases of
Geobacillus stearothermophilus.

| | GenBank Amino | GenBank Nucleotide |
|---|---|---|
| | Acid Sequence | Sequence |
| Species | Accession No. | Accession No. |
| Geobacillus | AAN81911 | AY186197.1, Region: |
| stearothermophilus | (SEQ ID NO: 9) | 1742 . . . 2485 |
| | | (SEQ ID NO: 10) |
| | AAN81912 | AY186197.1, Region: 1 . . . 531 |
| | (SEQ ID NO: 11) | (SEQ ID NO: 12) |
| | AAN81910 | AY186196.1, Region: |
| | (SEQ ID NO: 13) | 3549 . . . 5045 |
| | | (SEQ ID NO: 14) |
| | ACA01541 | DQ146476.2, |
| | (SEQ ID NO: 15) | Region: 13137 . . . 13817 |
| | | (SEQ ID NO: 16) |

Furthermore, Table 3 lists some illustrative examples of carboxylesterases from animals, insects and plants, and the GenBank Accession Numbers of their corresponding nucleotide and amino acid sequences.

TABLE 3
Illustrative examples of carboxylesterases of different species.

| | | | GenBank |
|---|---|---|---|
| | | GenBank Amino | Nucleotide |
| | | Acid Sequence | Sequence Accession |
| | Species | Accession No. | No. |
| Animal | Homo sapiens | AAA83932 | M65261 Region: |
| | | (SEQ ID NO: 17) | 1 . . . 1367 |
| | | | (SEQ ID NO: 18) |
| | Mus musculus | CAA73388 | Y12887 Region: |
| | (mouse) | (SEQ ID NO: 19) | 42 . . . 1739 |
| | | | (SEQ ID NO: 20) |
| | Xenopus laevis (frog) | NP_001080853 | NM_001087384 |
| | | (SEQ ID NO: 21) | Region: 38 . . . 1699 |
| | | | (SEQ ID NO: 22) |
| | Gallus gallus | NP_001013015 | NM_001012997 |
| | (chicken) | (SEQ ID NO: 23) | Region: 15 . . . 1685 |
| | | | (SEQ ID NO: 24) |
| Insect | Drosophila | AAA28520 | M33780 Region: |
| | melanogaster (fly) | (SEQ ID NO: 25) | join (3052 . . . 4438, |
| | | | 4495 . . . 4742) |
| | | | (SEQ ID NO: 26) |
| | Bombyx mori | NP_001037027 | NM_001043562 |
| | (silkworm) | (SEQ ID NO: 27) | Region: 33 . . . 1745 |
| | | | (SEQ ID NO: 28) |
| Plant | Arabidopsis thaliana | NP_176139 | NM_104623 |
| | | (SEQ ID NO: 29) | Region: |
| | | | 112 . . . 1194 |
| | | | (SEQ ID NO: 30) |
| | Malus pumila (apple) | ABB89007 | DQ279908 Region: |
| | | (SEQ ID NO: 31) | 79 . . . 981 |
| | | | (SEQ ID NO: 32) |

Variants of carboxylesterases may be generated by conservative substitutions to the wild type carboxylesterases, wherein a substituent amino acid has similar structural or chemical properties to the native amino acid, for example, similarity in polarity, charge, solubility, hydrophobicity, hydrophilicity, and/or the amphipathic nature of the residues. The variants may also be made by non-conservative substitutions or other changes to the amino acid sequence of the wild type carboxylesterase as long as the variants retain the carboxylesterase activity. Guidance in determining which and how many amino acid residues may be substituted, inserted or deleted without abolishing functional or biological activity may be found using computer programs well known in the art, for example, STAR software (see Bauer et al, STAR: predicting recombination sites from amino acid sequence, BMC Bioinformatics, 2006, vol 7, 437).

In certain embodiments, the present disclosure provides carboxylesterases and variants thereof that share at least 70%, 80%, 85%, 90%, 95%, 96%, 97%, 98%, or 99% amino acid sequence identity with one or more of the amino acid sequences of the carboxylesterases set forth in SEQ ID NOs: 9, 11, 13 and 15.

“Percent (%) amino acid sequence identity” with respect to the carboxylesterase polypeptide sequences identified herein is defined as the percentage of amino acid residues in a candidate sequence that are identical with the amino acid residues in the specific carboxylesterase polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In certain embodiments, the present disclosure provides nucleotide sequences encoding for a carboxylesterase or its variant that share at least 70%, 80%, 85%, 90%, 95%, 96%, 97%, 98%, or 99% sequence identity with one or more of the nucleotide sequences of the carboxylesterase set forth in SEQ ID NOs: 10, 12, 14, and 16.

“Percent (%) nucleotide sequence identity” with respect to carboxylesterase-encoding nucleotide sequences identified herein is defined as the percentage of nucleotides in a candidate sequence that are identical with the nucleotides in the carboxylesterase nucleotide sequence of interest, after aligning the sequences and introducing gaps, if necessary, to achieve the maximum percent sequence identity. Alignment for purposes of determining percent nucleotide sequence identity can be achieved in various ways that are within the skill in the art, for instance, using publicly available computer software such as BLAST, BLAST-2, ALIGN or Megalign (DNASTAR) software.

In one embodiment, percent amino acid sequence identity and percent nucleotide sequence identity may be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62. In situations where NCBI-BLAST2 is employed for amino acid (or nucleotide) sequence comparisons, the % amino acid (or nucleotide) sequence identity of a given amino acid sequence A (or a given nucleotide sequence A) to, with, or against a given amino acid sequence B (or a given nucleotide sequence B) (which can alternatively be phrased as a given amino acid sequence A (or a given nucleotide sequence A) that has or comprises a certain % amino acid (or nucleotide) sequence identity to, with, or against a given amino acid sequence B (or a given nucleotide sequence B)) is calculated as follows: 100 times the fraction X/Y, where X is the number of amino acid (or nucleotide) residues scored as identical matches by the sequence alignment program NCBI-BLAST2 in that program's alignment of A and B, and where Y is the total number of amino acid (or nucleotide) residues in B. It will be appreciated that where the length of sequence A is not equal to the length of sequence B, the % sequence identity of A to B will not equal the % sequence identity of B to A.

In another embodiment, percent amino acid sequence identity and percentage nucleotide sequence identity values may also be obtained as described below by using the WU-BLAST-2 computer program (Altschul et al., Methods in Enzymology 266:460-480 (1996)). All of the WU-BLAST-2 search parameters are set to the default values. When WU-BLAST-2 is employed, the % amino acid (or nucleotide) sequence identity of a given amino acid sequence A (or nucleotide sequence A) to, with, or against a given amino acid sequence B (or a given nucleotide sequence B) is determined by dividing (a) the number of matching identical amino acid (or nucleotide) residues between the sequence A and sequence B as determined by WU-BLAST-2 by (b) the total number of residues of sequence B, and (c) multiplied by 100.

In certain embodiments, the present disclosure provides thermostable carboxylesterases. The term “thermostable carboxylesterase” as used herein refers to a carboxylesterase capable of maintaining detectable enzymatic activity to hydrolyze carboxylic ester groups after being exposed to an elevated temperature at or above about 40° C. for a period of exposure time. Enzymatic activity of carboxylesterase may be detected using any method known in the art, for example, by measuring disappearance of a substrate or formation of a product under a given set of reaction conditions. Illustrative methods for detecting the enzymatic activity of carboxylesterase are spectroscopic methods, radiometric methods, colorimetric methods or high performance liquid chromatography based methods. Illustrative examples of substrates of carboxylesterases are, naphthyl acetate (NA), p-nitrophenyl acetate (p-NPA), methylthiobutyrate (MtB), or ¹⁴C-labelled esters. Control enzymatic activity may be measured using the same method under the same condition but in the absence of carboxylesterase. Enzymatic activity of carboxylesterase is considered detectable if it has a numerical value larger than the control enzymatic activity.

In certain embodiments, the elevated temperature is between about 40° C. and about 100° C., or between about 50° C. and about 90° C., or between about 50° C. and about 70° C. In certain embodiments, the elevated temperature is about 50° C., about 55° C., about 60° C., about 65° C., about 70° C., about 75° C., about 80° C., about 85° C., or about 90° C. The period of exposure time during which the carboxylesterase may be exposed to the elevated temperature may be determined by a person skilled in the art. In certain embodiments, the exposure time is up to 10 minutes, 30 minutes, 1 hour, 2 hours, 3 hours, 4 hours, 5 hours, 6 hours, 12 hours, 1 day, 2 days, 3 days, 4 days, 5 days, 6 days, 7 days, 8 days, 9 days, or 10 days. In certain embodiments, the exposure time is between 30 minutes and 10 days, or between 30 minutes and 5 days, or between 30 minutes and 1 day, or between 30 minutes and 6 hours, or between 30 minutes and 2 hours, between 1 hour and 2 hours, or between 10 minutes and 30 minutes.

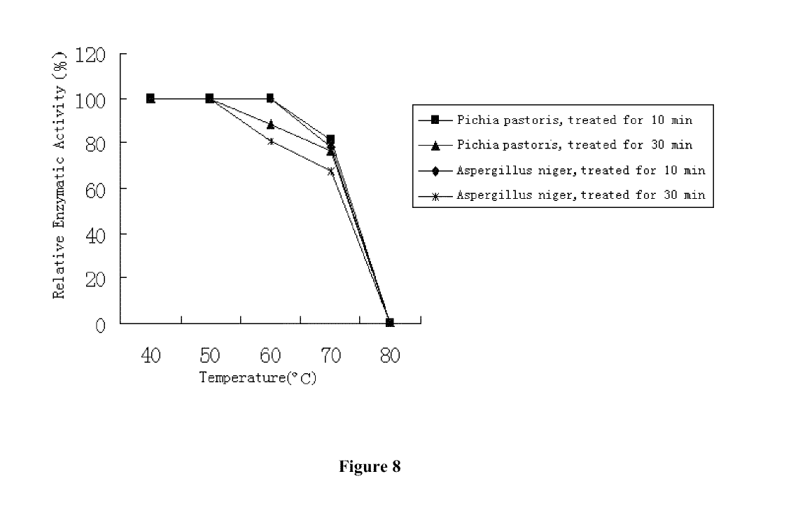

In certain embodiments, the present disclosure provides thermostable carboxylesterases of Geobacillus stearothermophilus having the amino acid sequences set forth in SEQ ID NOs: 9, 11, 13, and 15. As shown in FIG. 8, the enzymatic activity of thermostable carboxylesterase of SEQ ID NO: 9 may be measured after the enzyme has been exposed to elevated temperatures and times such as but not limited to, 40° C., 50° C., 60° C., 70° C., and 80° C., respectively, for 10 minutes or 30 minutes. The enzymatic activity of the carboxylesterase exposed and tested at 37° C. is also measured as a standard reference. The samples and the standard reference are otherwise tested and measured under the same conditions. After exposure at 60° C. for 10 minutes, the carboxylesterase may have almost 100% of the enzymatic activity of the standard reference. After exposure at 70° C. for 30 minutes, the carboxylesterase may have above 60% of the enzymatic activity of the standard reference.

### Expression Vectors and Host Cells

In one aspect, the present disclosure provides eukaryotic cells engineered to express a gene encoding for a carboxylesterase or its variant.

The term “express” or “expression” as used herein includes one or more steps involved in the production of the carboxylesterase including, but not limited to, transcription, post-transcriptional modification, translation, post-translational modification, and secretion. The term “engineered to express” as used herein refers to one or more steps of enabling a host cell to express a carboxylesterase or its variant in such a manner that is not naturally found in the host cell. In certain embodiments, the term “engineered to express” includes one or more steps of introducing an exogenous gene encoding for a carboxylesterase or its variant into a host cell for the purpose of expressing the carboxylesterase or its variant in the host cell. In certain embodiments, a host cell is engineered to express a mutated carboxylesterase or a carboxylesterase with mutated regulatory sequences by exposing the host cell to mutagens.

The term “gene” as used herein refers to polyribonucleotides or polydeoxyribonucleotides or mixed polyribo-polydeoxyribonucleotides that contain information encoding for a peptide or polypeptide. This includes single- and double-stranded molecules, i.e., DNA-DNA, DNA-RNA and RNA-RNA hybrids, as well as “protein nucleic acids” (PNA) formed by conjugating bases to an amino acid backbone. Genes include naturally-occurring polynucleotides or synthetic polynucleotides formed from naturally-occurring bases or modified bases. The term gene also encompasses the coding regions of a structural gene and sequences located adjacent to the coding regions on both the 5′ and 3′ ends that are useful for the transcription or translation of the RNA or polypeptide as well as intervening sequences (introns) between individual coding segments (exons).

The term “peptide” or “polypeptide” refers to amino acids linked to each other by peptide bonds or modified peptide bonds, i.e., peptide isosteres, and may contain modified amino acids other than the 20 naturally occurring amino acids. The term “peptide” or “polypeptide” also includes peptides or polypeptide fragments, motifs and the like, glycosylated peptides or polypeptides, and other modified peptides or polypeptides.

The term “encoding for” as used herein means being capable of being transcribed into mRNA and/or translated into a peptide or protein.

In certain embodiments, genes encoding for a carboxylesterase or its variants are inserted into expression vectors for expression by host cells.

The term “expression vector” as used herein refers to a nucleotide vehicle into which a gene encoding for a peptide or protein is operably inserted so that the encoded peptide or protein can be expressed. Illustrative examples of nucleotide vehicles that may be used to build expression vectors include, but are not limited to, plasmids, phagemids, cosmids, artificial chromosomes such as yeast artificial chromosome, bacterial artificial chromosome, or P1-derived artificial chromosome, bacteriaophages such as lambda phage or M13 phage, animal viruses such as retrovirus, adenovirus or papovavirus, and plant viruses such as potato virus X. Many eukaryotic expression vectors are commercially available. Selection of appropriate expression vectors is within the knowledge of those skilled in the art.

In certain embodiments, the expression vector is a vector suitable for expression in yeast cells. Illustrative examples are, pPIC3K (Invitrogen, Carlsbad, Calif.), pPIC9K (Invitrogen), pAO815 (Invitrogen), pGAPZ (Invitrogen), pYC2/CT (Invitrogen), pYD1 yeast display vector (Invitrogen), pESC vectors (Stratagene, La Jolla, Calif.), pESC-HIS vector (Stratagene), and pHIPX4 (Gietl et al, Mutational analysis of the N-terminal topgenic signal of watermelon glyoxysomal malate dehydrogenase using the heterologous host Hansenula polymorphs. Proc. Natl. Acad. Sci. USA 1994, vol 91, 3151-3155). In certain embodiments, the expression vector is a plasmid suitable for expression in yeast cells. In certain embodiments, the expression vector is a plasmid suitable for expression in Pichia pastoris. In certain embodiments, the expression vector is pPIC9K.

In certain embodiments, the expression vector is a vector suitable for expression in filamentous fungal cells. Illustrative examples are, pPTR (TaKaRa Bio Inc., Shiga, Japan), pDG1 (ATCC Catalog No. 53005), pAB366 (ATCC Catalog No. 77134), pAB520 (ATCC Catalog No. 77137), plasmid pYG1.2 (Liu et al, Construction of recombinant expression plasmid for Aspergillus niger, Journal of Tongji University (Medical science) 2001, vol 22, 1-3), pTAex3 (Sakuradani et al, D6-Fatty acid desaturase from an arachidonic acid-producing Mortierella fungus Gene cloning and its heterologous expression in a fungus, Aspergillus, Gene 1999, vol 238, 445-453), pSa123 (Gomi et al., Integrative transformation of Aspergillus oryzae with a plasmid containing the Aspergillus nidulans argB gene, Agric. Biol. Chem. 1987, vol 51, 2549-2555), pNAN8142 (Hiroyuki et al., Expression of Aspergillus oryzae Phytase Gene in Aspergillus oryzae RIB40 niaD, Journal of bioscience and bioengineering, 2006, Vol 102, No. 6, 564-567). In certain embodiments, the expression vector is a plasmid suitable for expression in filamentous fungal cells. In certain embodiments, the expression vector is a plasmid suitable for expression in Aspergillus niger. In certain embodiments, the expression vector is pYG1.2. An Escherichia coli DH5α strain containing the pYG1.2 plasmid (Escherichia coli DH5α/pYG1.2) was deposited with the China Center for Type Culture Collection (CCTCC), Wuhan University, Wuhan, China, on Jul. 27, 2009, and assigned the Accession No. CCTCC M 209165, under the terms and conditions of the Budapest Treaty.

In another aspect, the present disclosure provides an expression vector comprising a gene encoding for a carboxylesterase from a microbe or its variant, and a regulatory sequence capable of promoting expression of the carboxylesterase or its variant in a eukaryotic cell, wherein the regulatory sequence is operably linked to the gene. In certain embodiments, the amino acid sequences of the carboxylesterase or its variant have at least 70% sequence identity to the amino acid sequences of SEQ ID NOs: 9, 11, 13 or 15. In certain embodiments, the amino acid sequences of the carboxylesterase or its variant have at least 90% sequence identity to the amino acid sequences of SEQ ID NOs: 9, 11, 13 or 15. In certain embodiments, the nucleotide sequences of the carboxylesterase or its variant have at least 70% sequence identity to the nucleotide sequences of SEQ ID NOs: 10, 12, 14 or 16. In certain embodiments, the nucleotide sequences of the carboxylesterase or its variant have at least 90% sequence identity to the nucleotide sequences of SEQ ID NOs: 10, 12, 14 or 16.

In another aspect, the present disclosure provides an expression vector comprising a gene encoding for a carboxylesterase or its variant, and a regulatory sequence capable of promoting expression of the carboxylesterase or its variant in a filamentous fungal cell, wherein the regulatory sequence is operably linked to the gene.

The term “regulatory sequence” includes any component which is necessary or advantageous for the expression of a carboxylesterase or its variant of the present disclosure. Such regulatory sequences may include, but are not limited to, a promoter sequence, a transcription terminator, a leader sequence, and a polyadenylation sequence. The term “operably linked” means that the gene sequence is directly or indirectly linked to or associated with one or more regulatory sequence in a manner that allows expression of the gene encoding for the carboxylesterase or its variant. The gene coding sequence and the one or more regulatory sequences may be located on the same polynucleotide molecule and positioned in such a manner that allow expression of the gene encoding for the carboxylesterase or its variant. The gene coding sequence and the one or more regulatory sequences may be located on different polynucleotide molecules but the regulatory sequences can function to affect expression of the gene encoding for the carboxylesterase or its variant.

The regulatory sequence may contain an appropriate promoter sequence. As used herein, a “promoter sequence” refers to a segment of DNA that controls transcription of a DNA sequence to which it is operably linked. The promoter sequence includes specific sequences that are sufficient for RNA polymerase recognition, binding and transcription initiation. In addition, the promoter sequence may include sequences that modulate this recognition, binding and transcription initiation activity of RNA polymerase. These sequences may affect transcription on the same molecule or a different molecule. Functions of the promoter sequences, depending upon the nature of the regulation, may be constitutive or inducible by a stimulus. Any promoter sequence suitable for transcription control in eukaryotic cells may be used. In certain embodiments, the promoter sequence is suitable for transcription control in yeast cells and/or filamentous fungal cells. Illustrative examples of suitable promoter sequences in yeast cells are TEF promoter, CYC promoter, ADH1 promoter, 3-phosphoglycerate kinase promoter, glyceraldehyde-3-phosphate dehydrogenase (GAFDH or GAP) promoter, galactokinase (GAL1) promoter, galactoepimerase promoter, and alcohol dehydrogenase (ADH1) promoter. Illustrative examples of suitable promoter sequences in filamentous fungal cells are α-amylase promoter, glucoamylase promoter, alcohol dehydrogenase promoter (Kinghorn et al., Applied molecular genetics of filamentous fungi, Springer press, 1992, p 18). In certain embodiments, the promoters are inducible promoters that can turn on or off the carboxylesterase expression in response to a chemical or physical stimulus. Illustrative examples of inducible promoters are AOX1 promoter (inducible by methanol), GAL1 promoter (inducible by galactose), CUP promoter (inducible by Cu²⁺) (Wei Xiao, Yeast protocols, Edition: 2, Humana Press, 2005, p 320), and alc A promoter (inducible by alcohols).

The regulatory sequence may contain a suitable transcription terminator sequence, which is a sequence recognized by a eukaryotic cell RNA polymerase to terminate transcription. The terminator sequence may be operably linked to the 3′ terminus of the nucleotide sequence encoding for a carboxylesterase or its variants. Any terminator sequence which is functional in eukaryotic cells may be used in the present disclosure. In certain embodiments, the terminator sequence may be nucleotide sequence having transcription termination activity in yeast cells or filamentous fungal cells.

The regulatory sequence may also contain a suitable leader sequence, which is a non-translated region of an mRNA which is important for translation by eukaryotic cells. The leader sequence may be operably linked to the 5′ terminus of the nucleotide sequence encoding the carboxylesterase or its variants. Any leader sequence which is functional in eukaryotic cells may be used in the present disclosure. In certain embodiments, the leader sequence may be a nucleotide sequence functional in yeast cells or filamentous fungal cells.

The regulatory sequence may also contain a polyadenylation sequence, a sequence which may be operably linked to the 3′ terminus of the carboxylesterase gene sequence and which, when transcribed, is recognized by eukaryotic cells as a signal to add polyadenosine residues to the transcribed mRNA. Any polyadenylation sequence which is functional in eukaryotic cells may be used in the present disclosure. In certain embodiments, the polyadenylation sequence is functional in yeast cells or filamentous fungal cells.

In another aspect, the present disclosure provides an expression vector optionally further comprising a marker gene for selective identification of the expression vector. A marker gene is a gene encoding for a protein that can serve as a selection marker for identifying cells comprising the gene. Typical marker genes encode proteins that have one or more of the following characteristics: i) confer resistance to antibiotics or other toxic substances, e.g., Ampicillin, neomycin, methotrexate, etc.; ii) complement auxotrophic deficiencies, and iii) supply critical nutrients not available from the media. Marker genes may be inducible or non-inducible and will generally allow for positive selection. Suitable marker genes for yeast host cells include, but are not limited to, Ade2, His3, Leu2, Lys2, Met3, Trp1, Ura3, and neomycin or Kanamycin or Ampicillin resistance gene. Suitable marker genes for use in filamentous fungal host cells include, but are not limited to, amdS (acetamidase), argB (ornithine carbamoyltransferase), bar (phosphinothricin acetyltransferase), hygB (hygromycin phosphotransferase), niaD (nitrate reductase), pyrG (orotidine-5′-phosphate decarboxylase), sC (sulfate adenyltransferase), trpC (anthranilate synthase), as well as equivalents thereof.

In another aspect, the present disclosure provides a method of producing a carboxylesterase or its variant, wherein the expressed carboxylesterase or its variant is secreted to the outside of the eukaryotic cell. In certain embodiments, the gene encoding for the carboxylesterase or its variant is linked to a signal sequence that codes for a signal peptide. As used herein, the term “signal peptide” refers to an amino acid sequence that is linked to the carboxylesterase or its variant and that enables the expressed carboxylesterase or its variant to be transported/secreted outside of a cell membrane. In certain embodiments, the signal peptide may be linked to the amino terminus of the carboxylesterase or its variant. In certain embodiments, the signal peptide may be cut by a peptidase to remove the signal peptide from the carboxylesterase or its variant.

The signal sequence may be one or more naturally existing signal sequences of carboxylesterases, or foreign signal sequences added to the carboxylesterases. In certain embodiments, the signal sequences are functional in yeast cells and/or filamentous fungal cells. Illustrative examples of signal peptides functional in yeast cells are chicken lysozyme signal peptide (CLSP), signal peptide for Saccharomyces cerevisiae alpha-factor and signal peptide for Saccharomyces cerevisiae invertase. Illustrative examples of signal peptides functional in filamentous fungal cells are signal peptide for Aspergillus oryzae TAKA amylase, signal peptide for Aspergillus niger neutral amylase, signal peptide for Aspergillus niger glucoamylase, signal peptide for Rhizomucor miehei aspartic proteinase, signal peptide for Humicola insolens cellulase, and signal peptide for Humicola lanuginosa lipase.

The term “host cell” as used herein refers to a cell that is susceptible to transformation, transfection, transduction, and the like with an expression vector.

The expression vector may be introduced into the eukaryotic cells using any suitable methods known in the art, including without limitation, electroporation, calcium chloride-, lithium chloride-, lithium acetate/polyethylene glycol-, calcium phosphate-, DEAE-dextran-, liposome-mediated DNA uptake, spheroplasting, injection, microinjection, microprojectile bombardment, phage infection, viral infection, or other established methods. Alternatively, expression vectors containing the gene sequences of interest can be transcribed in vitro, and the resulting mRNA may be introduced into the host cell for transient expression by well-known methods, e.g., by injection (see, Kubo et al., Location of a region of the muscarinic acetylcholine receptor involved in selective effector coupling, FEBS Letts. 1988, vol 241, 119).

In certain embodiments, expression vectors may be introduced into yeast cells by methods such as protoplast transformation (see, e.g., Spencer et al, Genetic manipulation of non-conventional yeasts by conventional and non-conventional methods. J Basic Microbiol., 1988, vol 28, No. 5, 321-333), competent cells transformation (see, e.g., Gietz et al, Frozen competent yeast cells that can be transformed with high efficiency using the LiAc/SS carrier DNA/PEG method, Nat. Protoc. 2007; 2(1):1-4), electroporation (see, e.g., Suga et al, High-efficiency electroporation by freezing intact yeast cells with addition of calcium, Curr Genet., 2003, vol 43, No. 3, 206-211), or conjugation through cell-to-cell contact (see, e.g., Nishikawa et al, Trans-kingdom conjugation offers a powerful gene targeting: tool in yeast, 1998, Genetic Analysis: Biomolecular Engineering, vol 14, No. 3, 65-73).

In certain embodiments, the expression vector may be introduced into filamentous fungal cells by protoplast transformation comprising steps of protoplast isolation, regeneration, and fusion (see, Arora et al, Handbook of fungal biotechnology, 2nd Edition, CRC Press, 2004, p 9-24). Suitable procedures for transformation of filamentous fungal cells are described in various publications (see, for example, Ruiz et al, Strategies for the transformation of filamentous fungi, J Appl Microbiol., 2002, vol 92, No. 2, 189-195; Hynes et al, Genetic transformation of filamentous fungi, Journal of Genetics, 1996, vol 75, No. 3, 297-311).

In another aspect, the present disclosure provides a eukaryotic cell comprising an expression vector, wherein the expression vector containing a gene encoding for a carboxylesterase from a microbe or its variant, and a regulatory sequence capable of promoting expression of the carboxylesterase or its variant in a eukaryotic cell, wherein the regulatory sequence is operably linked to the gene. In certain embodiments, the eukaryotic cell is yeast cell. In certain embodiments, the eukaryotic cell is Pichia pastoris.

In another aspect, the present disclosure provides a eukaryotic cell comprising an expression vector containing a gene encoding for a carboxylesterase or its variant, and a regulatory sequence capable of promoting expression of the carboxylesterase or its variant in a filamentous fungal cell, wherein the regulatory sequence is operably linked to the gene. In certain embodiments, the filamentous fungal cell is Aspergillus niger.

### Cell Culturing

The eukaryotic cells engineered to express a carboxylesterase or its variant of the present disclosure may be cultured in any suitable medium under conditions suitable for expression of the carboxylesterase or its variant. For example, the cells may be cultivated by shake flask cultivation, small-scale or large-scale fermentation (including continuous, batch, fed-batch, or solid state fermentations) in laboratory or industrial fermentors. The cultivation may take place in a suitable nutrient medium comprising carbon and nitrogen sources and inorganic salts. Suitable media are available from commercial suppliers or may be prepared using commercially available ingredients.

The cultivation conditions such as temperature, pH, incubation time and presence of an inducer may be adjusted to allow higher expression of the carboxylesterases. Cultivation conditions may be adjusted by people skilled in the art. In certain embodiments, cultivation conditions may be determined by cultivating cells engineered to express a carboxylesterase or its variant under a wide range of conditions, measuring the expression of the carboxylesterase or its variant and selecting the cultivation conditions that allow a relatively high level expression of the carboxylesterase or its variant.

Suitable temperature, pH, and incubation time for cell cultivation usually depend on the host cells. In certain embodiments, the cultivation temperature may range from about 20° C. to about 80° C., from about 30° C. to about 70° C., from about 30° C. to about 60° C., from about 30° C. to about 50° C., or from about 30° C. to about 40° C. In certain embodiments, the cultivation temperature is at about 20° C., about 25° C., about 30° C., about 35° C., about 37° C., about 40° C., about 50° C., about 60° C., about 70° C., or about 80° C.

In certain embodiments, the cultivation pH may range from about 2 to about 8.5, from about 3 to about 8.5, from about 4 to about 8.5, from about 5 to about 8.5, from about 6 to about 8.5, or from about 7 to about 8.5. In certain embodiments, the cultivation pH is at about 2, about 3, about 4, about 5, about 6, about 7, about 8, or about 8.5.

In certain embodiments, the incubation time may be at least one day, at least 2 days, at least 3 days, or at least 4 days. In certain embodiments, the incubation time may range from 1 day to 10 days, 2 days to 9 days, 3 days to 8 days, or 4 days to 7 days. In certain embodiments, the incubation time is 1 day, 2 days, 3 days, 4 days, 5 days, 6 days, 7 days, 8 days, 9 days, or 10 days.

In certain embodiments, the eukaryotic cells may be cultured in the presence of an inducer that can induce the expression of the carboxylesterase or its variant. The selection of an inducer may be based on the inducible promoter operably linked to the gene encoding for the carboxylesterase or its variant. In an illustrative embodiment, the eukaryotic cells are cultivated in the presence of methanol to induce AOX1 promoter operably linked with the gene encoding the carboxylesterase or its variant. In another illustrative embodiment, the eukaryotic cells are cultivated in the presence of galactose to induce GAL1 promoter. In another illustrative embodiment, the eukaryotic cells are cultivated in the presence of Cu²⁺ to induce CUP promoter. In another illustrative embodiment, the eukaryotic cells are cultivated in the presence of one or more alcohols to induce alc A promoter. The amount of an inducer in the cell culture may be adjusted by people skilled in the art to allow a relatively higher level expression of the carboxylesterase or its variant.

The expression level of a carboxylesterase or its variant may be determined using well-established techniques known in the art. In certain embodiments, the expression level of a carboxylesterase or its variant is measured by quantifying the amount of mRNA transcribed or the amount of protein translated.

In an illustrative embodiment, mRNA levels can be determined by Northern blot analysis (Alwine et al., Method for detection of specific RNAs in agarose gels by transfer to diazobenzyloxymethyl-paper and hybridization with DNA probes, Proc. Natl. Acad. Sci. USA 1977, vol 74, 5350-5354; Bird, Size separation and quantification of mRNA by northern analysis, Methods Mol. Biol. 1998, vol 105, 325-36). Briefly, poly(A) RNA is isolated from cells, separated by gel electrophoresis, blotted onto a support surface (e.g., nitrocellulose or Immobilon-Ny transfer membrane (Millipore Corp., Bedford, Mass.)), and incubated with a labeled (e.g., fluorescently labeled or radiolabeled) oligonucleotide probe that is capable of hybridizing with the mRNA of interest. In another illustrative embodiment, mRNA levels can be determined by quantitative RT-PCR (for review, see Freeman et al., Quantitative RT-PCR: pitfalls and potential, Biotechniques 1999, vol 26, 112-122) or semi-quantitative RT-PCR analysis (Ren et al., Lipopolysaccharide-induced expression of IP-10 mRNA in rat brain and in cultured rat astrocytes and microglia, Mol. Brain. Res. 1998, vol 59, 256-263). In accordance with this technique, poly(A) RNA is isolated from cells, used for cDNA synthesis, and the resultant cDNA is incubated with PCR primers that are capable of hybridizing with the template and amplifying the template sequence to produce levels of the PCR products that are proportional to the cellular levels of the mRNA of interest.

In an illustrative example, the expressed carboxylesterase or its variant is detected by electrophoresis such as sodium dodecyl sulfate polyacrylamide gel electrophoresis (SDS-PAGE), the density of a carboxylesterase band on SDS-PAGE gel may be scanned to quantify the protein using a commercial scanner (for example, GS-800 densitometer of Bio-Rad). In another illustrative example, the expressed carboxylesterase or its variant is detected by Western blot analysis using antibodies specifically recognizing the carboxylesterase or its variant. In another illustrative example, the expressed carboxylesterase or its variant is detected by measuring their enzymatic activity using a substrate.

Enzymatic activity of the expressed carboxylesterase may be determined using methods known in the art. The enzymatic activity may be characterized by measuring the disappearance of a substrate or the formation of a product. The measurement may be spectroscopic, radiometric, colorimetric or based on high performance liquid chromatography. Any substrate suitable for a carboxylesterase reaction may be used. Illustrative examples of substrates of carboxylesterases are, naphthyl acetate (NA), p-nitrophenyl acetate (p-NPA), methylthiobutyrate (MtB), or ¹⁴C-labelled esters. In an illustrative embodiment, the enzymatic activity of carboxylesterases may be quantified by spectroscopic measurement of a complex formed between the chromogenic reagent Fast Blue B salt and α-naphthol, which is the product of hydrolysis by a carboxylesterase of the substrate α-naphthyl acetate.

In another aspect, the present disclosure provides a method of producing a carboxylesterase or its variant, wherein the carboxylesterase or its variant is produced in an amount of at least about 12 mg/L and up to 100 mg/L. In certain embodiments, the yield of carboxylesterase or its variant is at least 12 mg/L, or at least 15 mg/L, or at least 17 mg/L, or at least 19 mg/L. In certain embodiments, the yield of carboxylesterase or its variant is 1 mg/L to 100 mg/L, 10 mg/L to 100 mg/L, 15 mg/L to 100 mg/L, 20 mg/L to 100 mg/L, 30 mg/L to 100 mg/L, 10 mg/L to 50 mg/L, 15 mg/L to 50 mg/L, 20 mg/L to 50 mg/L, or 30 mg/L to 50 mg/L. In certain embodiments, the carboxylesterase or its variant is produced in an amount of about 1 mg/L, about 5 mg/L, about 10 mg/L, about 12 mg/L, about 15 mg/L, about 20 mg/L, about 30 mg/L, about 40 mg/L, about 50 mg/L, or about 100 mg/L.

### Isolation of the Expressed Carboxylesterases

The expressed carboxylesterase or its variant may be isolated from the cell culture using standard methods known in the art including, without limitation, centrifugation, filtration, extraction, spray-drying, evaporation, or precipitation (see I. M. Rosenberg (Ed.), Protein Analysis and Purification: Benchtop Techniques, 1996, Birkhauser, Boston, Cambridge, Mass.; Janson et al, Protein Purification, 1989, VCH Publishers, New York)).

The expressed carboxylesterase may be further purified by a variety of procedures known in the art including, but not limited to, chromatography (e.g., ion exchange, affinity, hydrophobic, chromatofocusing, and size exclusion), electrophoretic procedures (e.g., preparative isoelectric focusing, SDS-PAGE), and differential solubility (e.g., ammonium sulfate precipitation).

In certain embodiments, antibody-based methods can be used to isolate and purify expressed carboxylesterases or variants thereof. Antibodies that can bind to the carboxylesterases or variants thereof, can be produced and isolated using methods known and practiced in the art. Carboxylesterases or variants thereof can be purified from a cell lysate or from the supernatant of the culture medium by chromatography on antibody-conjugated solid-phase matrices such as immunoprecipitation (see Harlow et al, Using Antibodies: A Laboratory Manual, Cold Spring Harbor Laboratory, 1999, Cold Spring Harbor, N.Y.).

In another aspect, the present disclosure provides isolated carboxylesterases or variants thereof. “Isolated carboxylesterase” as used herein refers to a carboxylesterase that is substantially free of the other cellular components with which they are associated during the production methods described herein. “Substantially free” includes a preparation of carboxylesterase having less than about 50%, 40%, 30%, 20%, 10%, 5% or 1% (by dry weight) of the other cellular components or other contaminating materials that are not the carboxylesterase or its variant of interest. In certain embodiments, the isolated carboxylesterase has less than 50%, 40%, 30%, 20%, 10%, 5%, or 1% (by dry weight) of contaminating materials that are not the carboxylesterase or its variant of interest.

### Compositions Comprising Expressed Carboxylesterases and Uses Thereof

In another aspect, the present disclosure provides a composition comprising a eukaryotic cell and a carboxylesterase or its variant expressed by the eukaryotic cell. In certain embodiments, the present disclosure provides a composition comprising a eukaryotic cell and a microbial carboxylesterase or its variant expressed by the eukaryotic cell. In certain embodiments, the present disclosure provided a composition comprising a filamentous fungal cell and a carboxylesterase or its variant expressed by the filamentous fungal cell. In an illustrative embodiment, the composition is directly obtained from a cell culture containing eukaryotic cells engineered to express a gene encoding for a carboxylesterase or its variant. The cell culture may be filtered or centrifuged or otherwise treated to get rid of the culture medium, cell debris and/or other unwanted substances. The cell culture may undergo further purification processes as deemed suitable by a person skilled in the art to increase the concentration of the carboxylesterases therein. The composition may be prepared in liquid form or dried solid form.

In another aspect, the present disclosure provides a composition comprising isolated carboxylesterases or variants thereof produced by a method described herein. In certain embodiments, the composition comprising isolated carboxylesterases or variants thereof having no more than 50%, 40%, 30%, 20%, 10%, 5% or 1% (by dry weight) of contaminating materials that are not the carboxylesterase or its variant of interest.

The compositions of the present disclosure may be used in various biological, agricultural and pharmaceutical applications. The composition of the present disclosure may be used to convert a compound with a carboxyl ester group to a compound without a carboxyl ester group, comprising incubating the compound with a carboxyl ester group with a carboxylesterase or its variant. In an illustrative embodiment, the composition is used to convert a prodrug with a carboxyl ester group to a drug without a carboxyl ester group. In another illustrative embodiment, the composition is used to convert a pesticide with a carboxyl ester group to a detoxified pesticide without a carboxyl ester group.

In another aspect, the present disclosure provides a method of detoxifying pesticides comprising incubating pesticides with the composition provided herein. Pesticides, as used herein, refer to chemical agents used to kill, repel or act against pests such as insects, plant pathogens, and weeds. Some pesticides contain one or more carboxyl ester groups in the chemical structures. Hydrolysis of the carboxyl ester groups by a carboxylesterase can convert the toxic pesticides to non-toxic substances. This is useful for cleaning up unwanted pesticide residues on agricultural products such as vegetables and fruits or in the environment such as water and soil. It is also useful for reducing or eliminating the toxic effects of pesticides in the event of poisoning. Illustrative examples of pesticides that may be detoxified by carboxylesterases are organophosphate pesticides, carbamate pesticides and pyrethroid pesticides.

In certain embodiments, the composition may be provided in the form of cell extracts from or culture supernatants of host cells that are engineered to expresses the carboxylesterase or its variant, or may be provided in the form of isolated carboxylesterase or its variant. The amount of the composition to be used may be determined as needed by people practicing the method. In certain embodiments, the amount of the composition to be used may depend on the amount and type of pesticide contained in the sample to be detoxified, and the enzymatic activity of the composition to hydrolyze the specific pesticide. In an illustrative embodiment, 0.1 nmol carboxylesterase is incubated with a sample containing 1 nmol of a carboxyl ester group containing pesticide; the carboxylesterase degraded about 100% of the pesticide after incubation for 4 hours. In another illustrative embodiment, 0.1 nmol carboxylesterase is incubated with a sample containing 2 nmol of a carboxyl ester group containing pesticide; the carboxylesterase degraded about 85% of the pesticide after incubation for 6 hours.

The incubation time may be determined as needed by people practicing the method. In certain embodiments, the incubation time may range from about 1 hour to about 3 weeks, from about 3 hours to about 7 days, and from about 4 hours to about 3 days, for example, about 1 hour, about 1.5 hour, about 3 hours, about 6 hours, about 9 hours, about 1 day, about 3 days, about 7 days, about 9 days, about 12 days, about 15 days, and about 3 weeks. Other conditions for incubation, for example, temperature, pH, presence of co-factors may be selected by people skilled in the art to detoxify a higher percentage of the pesticide.

In certain embodiments, the amounts of pesticides in a sample under treatment may be reduced by at least 30%, at least 40%, at least 50%, at least 60%, at least 70%, at least 80%, or at least 90%. In certain embodiments, the amounts of pesticides in a sample under treatment may be reduced by 30% to 100%, 40% to 90%, 50% to 80%, or 60% to 70%. In certain embodiments, the amounts of pesticides in a sample under treatment may be reduced by 30%, 40%, 50%, 60%, 70%, 80%, 90%, 95%, 99%, and 100%.

In another aspect, the present disclosure provides a method of converting a prodrug into a drug comprising incubating the prodrug with the composition provided herein. Some prodrugs may contain carboxyl ester groups that make the prodrugs pharmaceutically inactive. A carboxylesterase described herein can hydrolyze the carboxyl ester groups and convert the inactive prodrug into the active drug. In an illustrative example, irinotecan, an anti-cancer prodrug, is converted by carboxylesterase into the active drug compound 7-ethyl-10-hydroxycamptothecin, a topoisomerase I inhibitor (Yoon et al, Activation of a camptothecin prodrug by specific carboxylesterases as predicted by quantitative structure-activity relationship and molecular docking studies, Mol Cancer Ther 2003, vol 2, 1171). In another illustrative example, prodrug oseltamivir is converted into the active drug oseltamivir carboxylate by carboxylesterase (Shi et al, anti-influenza viral prodrug oseltamivir is activated by carboxylesterase hcel and the activation is inhibited by anti-platelet agent clopidogrel, J. Phar. Exp. Ther. 2006, vol 319, 1477-1484).

### EXAMPLES

The following Examples are set forth to aid in the understanding of the present disclosure, and should not be construed to limit in any way the scope of the invention as defined in the claims which follow thereafter.

Materials and Culture Media

1% agarose gel: 400 mg agarose, 39.2 ml H₂O, 0.8 ml 50×TAE, and 1 μg ethidium bromide.

LB medium (1 L): 10 g tryptone, 5 g yeast extract, 10 g NaCl, pH 7.4.

LB plate: LB medium containing 1.5% agar.

RDB plate: 1 M sorbitol, 2% glucose, 1.34% Yeast Nitrogen Base (YNB), 2% agar, 4×10⁻⁵% biotin, and 0.005% amino acid.

YPD medium: 1% yeast extract, 2% tryptone and 2% glucose.

YPD plate: 1% yeast extract, 2% tryptone, 2% glucose, and 2% powdered agar.

BMMY medium: 1% yeast extract, 2% tryptone, 100 mM potassium phosphate buffer (pH 6.0), 1.34% YNB, 4×10⁻⁵% biotin and 0.5% methanol.

MN broth (400 ml): 16 ml MN salt solution (37.5 g sodium nitrate, 3.25 g chloride potassium, 9.5 g monopotassium phosphate in 250 ml H₂O), 0.4 ml trace elements (containing 2.2 g zinc sulfate heptahydrate, 1.1 g boric acid, 0.5 g manganese chloride tetrahydrate, 0.5 g ferrous sulfate heptahydrate, 0.17 g cobalt chloride hexahydrate, 0.16 g copper sulfate pentahydrate, 0.15 g sodium molybdate, 5 g disodium ethylenediaminetetraacetate per 100 ml, pH 6.5), 6 g glucose, 0.4 g casein acids hydrolysate, 8 ml 50×MgSO₄ solution (6.5 g MgSO₄.7H₂O in 250 ml H₂O), pH 6.5.

MN+URI broth (400 ml): 0.4 g uridine, 400 ml MN broth.

MN+SORB agar medium (1 L): 40 ml MN salt solution, 1 ml trace elements, 10 g glucose, 218.64 g sorbitol, 15 g agar, 20 ml 50×MgSO₄ solution, pH 6.5.

STC buffer (300 ml): 65.6 g sorbitol, 0.36 g Tris base, 2.2 g CaCl₂.2H₂O, pH 7.5.

PEG solution (100 ml): 60 g PEG6000, 0.12 g Tris, 0.74 g CaCl₂, pH 7.5.

NM buffer (500 ml): 29.25 g NaCl, 2.132 g MES, pH 5.8.

MM buffer (200 ml): 59.15 g MgSO₄.7H₂O, 0.8 g MES, pH 5.8.

6×SDS-PAGE loading buffer: 300 mM Tris-HCl (pH 6.8), 12% (w/v/) SDS, 0.6% (w/v) Bromophenol Blue, 60% (v/v) glycerol, 6% (w/v) β-Mercaptoethanol.

PBST buffer: 0.01 M phosphate-buffered saline, pH 7.2, supplemented with 0.1 (v/v) Tween 20.

MGY medium: 1.34% YNB, 1% glycerol, 4×10⁻⁵% biotin.

Enzyme dilution buffer (100 ml): 3.5 g NaCl, 0.11 g CaCl₂, 1 g glucose, pH 5.8.

Lywallzyme: 0.2 g Lywallzyme dissolved in enzyme dilution buffer.

BSA: 12 mg/ml BSA dissolved in enzyme dilution buffer.

12% polyacrylamide resolving gel (10 ml): 4 ml H₂O, 3.3 ml 30% polyacrylamide, 2.5 ml 1.5M Tris-HCl (pH 8.8), 0.1 ml 10% SDS, 0.1 ml 10% AP, 4 μl TEMED.

5% polyacrylamide stacking gel (5 ml): 3.44 ml H₂O, 0.83 ml 30% polyacrylamide, 0.63 ml 0.5M Tris-HCl (pH 6.8), 0.05 ml 10% SDS, 0.05 ml 10% AP, 4 μl TEMED.

All culture media and solutions are sterilized.

### Example 1

### Expression of Recombinant Carboxylesterase in Aspergillus niger M54 Strain

Carboxylesterase gene is amplified by PCR from Geobacillus stearothermophilus CICC 20156 strain (purchased from China Center of Industrial Culture Collection, Beijing, China), using the genomic DNA of the CICC 20156 strain as template and the following primers: forward primer P1 (SEQ ID NO: 33), and reverse primer P2 (SEQ ID NO: 34). The primers contain sequences from the carboxylesterase gene set forth in SEQ ID NO: 9. The forward primer contains an Xba I site and a KEX2 site while the reverse primer includes a HpaI site as well as nucleotides encoding for hexa histidine tag (His-tag, coding sequence of His-tag is marked with a wave underline), which consists of six histidine residues that can be used for affinity purification and antibody detection. The reverse primer contains the His-tag coding sequence linked to a portion of the nucleotide sequence of the 3′ end of the carboxylesterase gene. The PCR product generated using primers P1 and P2 is called the CarE-his gene (SEQ ID NO: 35) encoding for a fusion protein of carboxylesterase and a His-tag fused at the C terminal.

TABLE 4
Nucleotide acid sequences of primers
P1 and P2, and the CarE-his gene.

| Name | Sequence |
|---|---|
| P1 | 5′CGTCTAGAAAGAGAATGATGAAAATTGTTCCGCCG 3′ |
| | (SEQ ID NO: 33) |
| P2 | 5′CGGTTAACTTA [symbol] CCAATCTAA |
| | CGATTCAA3′ |
| | (SEQ ID NO: 34) |
| CarE- | 5′ATGATGAAAATTGTTCCGCCGAAGCGTTTTTCTTTGAA |
| his | GCCGGGGAGCGGGCGGTGCTGCTTTTGCATGGGTTTACCG |
| | GCAATTCCGCCGACGTTCGGATGCTTGGGCGATTCTTGGA |
| | ATCGAAAGGGTATACGTGCCACGCTCCGATTTACAAAGGG |
| | CATGGCGTGCCGCCGGAAGAGCTCGTCCACACCGGACCGG |
| | ATGATTGGTGGCAAGACGTCATGAACGGCTATCAGTTTTT |
| | GAAAAACAAAGGCTACGAAAAAATTGCCGTGGCTGGATTG |
| | TCGCTTGGAGGCGTATTTTCTCTCAAATTAGGCTACACTG |
| | TACCTACACAAGGCATTGTGACGATGTGCGCGCCGATGTA |
| | CATCAAAAGCGAAGAAACGATGTACGAAGGTGTGCTCGAG |
| | TATGCGCGCGAGTATAAAAAGCGGGAAGGGAAATCAGAGG |
| | AACAAATCGAACAGGAAATGGAACGGTTCAAACAAACGCC |
| | GATGAAGACGTTGAAAGCCTTGCAAGAACTCATTGCCGAT |
| | GTGCGCGCCCACCTTGATTTGGTTTATGCACCGACGTTCG |
| | TCGTCCAAGCGCGCCATGATGAGATGATCAATCCAGACAG |
| | CGCGAACATCATTTATAACGAAATTGATCGCCGGTCAAAC |
| | AAATCAAATGGTATGAGCAATCAGGCCATGTGATTACGCT |
| | TGATCAAGAAAAAGATCAGCTGCATGAAGATATTTATGCA |
| | TTTCTTGAATCGTTAGATTGG [symbol] T |
| | GA 3′ |
| | (SEQ ID NO: 35) |

The PCR is performed in a 50 μl reaction system containing the following composition: 5 μl 10×Pfu Buffer (Tiangen biotech Co. Ltd, Beijing, China), 4 μl dNTP mix (Tiangen biotech Co. Ltd, Beijing, China), 1 μl forward primer P1, 1 μl reverse primer P2, 1 μl template DNA, 1 μl pfu DNA polymerase (Tiangen biotech Co. Ltd, Beijing, China), and 37 μl double distilled H₂O. The following cycles are used in PCR: 95° C. for 5 minutes, followed by 30 cycles of 95° C. for 45 seconds, 60° C. for 45 seconds, and 72° C. for 90 seconds; and then 72° C. for 5 minutes. The PCR product is then put on electrophoresis in 1% agarose gel and the 780 bp band is cut and purified by gel extraction using a gel extraction kit (Tiangen biotech Co. Ltd, Beijing, China) according to the manufacturer's instructions.

The purified PCR product is inserted into pYG1.2 vector, which is constructed using methods previously published (Liu, Zhongbin, et al, Construction of recombinant expression plasmid for Aspergillus niger, Journal of Tongji University (medical science), 2001, 22, vol 3, 1-3). pYG1.2 vector contains a pyr gene from Aspergillus niger ATCC 12049 strain, a gla A coding sequence and regulatory sequences. The steps for making the pYG1.2 vector are described briefly below.

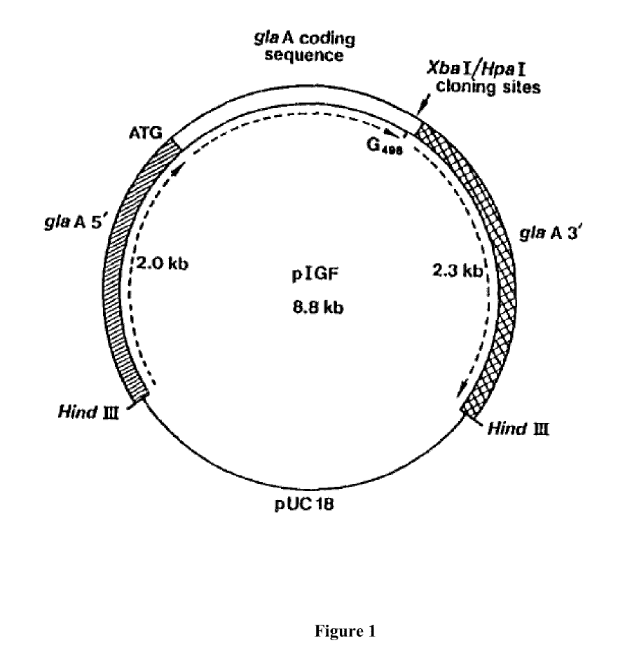

The commercially available plasmid pUC18 (Fermentas Inc., Burlington, Canada) is used to construct the pIGF vector. The pUC18 vector contains a beta-lactamase gene that confers resistance to Ampicillin. A 4.8 kb fragment, containing the gla A encoding sequence (encoding for the first 498 amino acids of glucoamylase), a 2.0 kb upstream regulatory sequence (the promoter of gla A) and a 2.3 kb downstream regulatory sequence (terminator sequence of gla A), are inserted into the pUC18 vector to make the pIGF vector. The gla A coding sequence is inserted to help increase the expression level of the target protein. FIG. 1 shows a schematic diagram of the structure of the pIGF vector. The inserted gla A coding sequence includes an Xba I/Hap I cloning site (shown in FIG. 1) that can be used to create fusion proteins with gla A.

Then the pIGF vector is inserted with pyr G gene of Aspergillus niger ATCC12049 strain to make the pYG1.2 vector. A 600 bp fragment containing the conservative sequences of pyrG gene is obtained by PCR using the pAB4.1 plasmid (provided by Institute of Food Research, Norwich, UK, which contains pyrG gene from Aspergillus niger ATCC9029 strain) as the template. The fragment is labeled with ³²P and is used as a probe in plaque hybridization (Sambrook, J, et al, A laboratory manual, New York: Cold Spring Harbor Laboratory Press, 1989). A 9.8 kb nucleotide fragment containing the pyrG gene is isolated from the gene library of Aspergillus niger ATCC 12049 strain, and is further digested with Xho I to obtain a 2.3 kb fragment, which is confirmed to contain the pyrG gene of ATCC 12049 strain by restriction zymography and PCR.

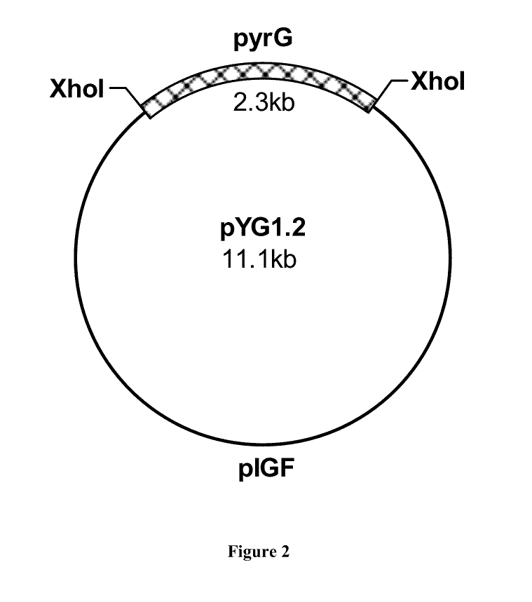

The obtained 2.3 kb fragment is ligated with the linearized fragment of pIGF vector digested with XhoI. The ligation product is transformed into Escherichia coli (E. coli) DH5α and clones grown on LB plates containing Ampicillin are picked. PCR is performed to identify positive clones containing the inserted pyr G gene. The identified positive clones are propagated to extract plasmids, and the recombinant plasmids are sequenced to confirm successful insertion of the pyrG gene. One of the confirmed plasmids is named as pYG1.2 and used as a vector in the following molecular cloning studies. A schematic map of pYG1.2 is shown in FIG. 2. An Escherichia coli DH5α strain containing the pYG1.2 plasmid (Escherichia coli DH5α/pYG1.2) is deposited with CCTCC, Wuhan University, Wuhan, China, on Jul. 27, 2009. The deposit No. is CCTCC M 209165. The deposit will be maintained under the terms and conditions of the Budapest Treaty. The plasmid pYG1.2 may be recovered from the Escherichia coli DH5α/pYG1.2 strain by conventional plasmid extraction methods.

To insert the carboxylesterase gene into the pYG1.2 vector, the purified CarE-his gene product and the pYG1.2 plasmid are separately digested using both XbaI and HpaI (New Englan Biolabs, Inc., Ipswich, Mass.), followed by electrophoresis in 1% agarose gel and purification by gel extraction. The digested CarE-his gene product and the digested pYG1.2 fragment are ligated using T4 ligase (Takara Bio. Inc., Japan).

E. Coli DH5α are transformed by mixing competent E. Coli DH5α cells with 20 μl ligation product, incubating on ice for 30 minutes, heat shock at 42° C. for 90 seconds, incubating on ice for 5 minutes, followed by addition of 200 μl LB medium and incubation in a shaker at 37° C. for 45 minutes. 50 μl of the resultant bacteria culture are inoculated onto LB plates containing Ampicillin, and are incubated overnight at 37° C.

Individual colonies of E. Coli grown on the LB plates containing Ampicillin are screened by PCR using the individual E. coli colonies as templates under the same PCR reaction conditions as described above. The PCR products are characterized by electrophoresis in 1% agarose gel, and positive colonies showing a band at 780 bp are propagated overnight in LB medium with Ampicillin, followed by plasmid purification using plasmid extraction kit (Tiangen biotech Co. Ltd, Beijing, China) according to the manufacturer's instructions.

The obtained recombinant plasmids are characterized by digestion using XbaI and HpaI followed by electrophoresis. The positive plasmids are sequenced and the results confirm successful insertion of the carboxylesterase gene into the pYG1.2 plasmid. The recombinant plasmid is named as pYG1.2-CarE-his and used in the subsequent studies.

Aspergillus niger M54 strain is used to express carboxylesterase. Aspergillus niger M54 is a pyr G deficient strain that cannot grow on uridine-free medium. Aspergillus niger M54 is obtained by exposing Aspergillus niger ATCC 12049 strain to UV irradiation and screen for strains deficient in pyr G gene as previously described (Liu, Zhongbin et al, Construction of pyr G auxotrophic Aspergillus niger strain, Journal of microbiology, 2001, 21, vol 3, 15-16). The strain has been deposited with CCTCC, Wuhan University, Wuhan, China, on Jun. 14, 2009. The deposit No is CCTCC M 209121. The deposit will be maintained under the terms and conditions of the Budapest Treaty.

The plasmid pYG1.2-CarE-his is used to transform the protoplasts of Aspergillus niger M54. Protoplasts are prepared as follows: 1 L flask containing 200 ml MN+URI broth is inoculated with Aspergillus niger M54 suspension at the cell density of 4×10⁸ and incubated in a shaker at 200 rpm at 30° C. for 24 hours; the treated cells are collected and 1 g of the cells (wet weight) is suspended with 10 ml ice cold MM buffer; 1 ml Lywallzyme and 0.5 ml BSA are added to the cell suspension following gentle shake at 30° C. at 80 rpm for 10 minutes and then at 50 rpm for 2 hours; the suspension is then centrifuged at 4° C. at 1500 rpm for 2 minutes and the supernatant is transferred and mixed with NM buffer followed by centrifugation at 4° C. at 2500 rpm for 10 minutes; the precipitates are mixed with ice cold STC buffer to a total volume of 50 ml followed by centrifugation at 4° C. at 2000 rpm for 10 minutes to yield milky white protoplasts which is then suspended in 1 ml ice cold STC buffer.

The protoplasts are transformed with pYG1.2-CarE-his plasmid, pYG1.2 plasmid (positive control) and blank buffer (negative control), respectively. 3.0 μg plasmid DNA is mixed with 100 μl protoplast suspension in a 15 ml tube and incubated at room temperature for 25 minutes. A total volume of 1250 μl PEG solution is added drop by drop into the mixture of plasmid and protoplast with gentle mix, followed by incubation at room temperature for 20 minutes. Ice cold STC buffer is added to fill the tube followed by gentle mix until the PEG solution is completely diluted. After centrifugation at 4° C. at 2000 rpm for 10 minutes, the cells are resuspended in 300 μl STC buffer, from which 100 μl is taken to be inoculated onto the uridine-free MN+SORB agar medium plates and incubated at 30° C. for 3-5 days to select transformants that can grow on the selective medium. The non-transformed Aspergillus niger M54 would not grow on the uridine-free medium while Aspergillus niger transformed with pYG1.2-CarE-his and pYG1.2 both grow on the uridine-free medium, indicating the plasmids are successfully expressed in Aspergillus niger M54.

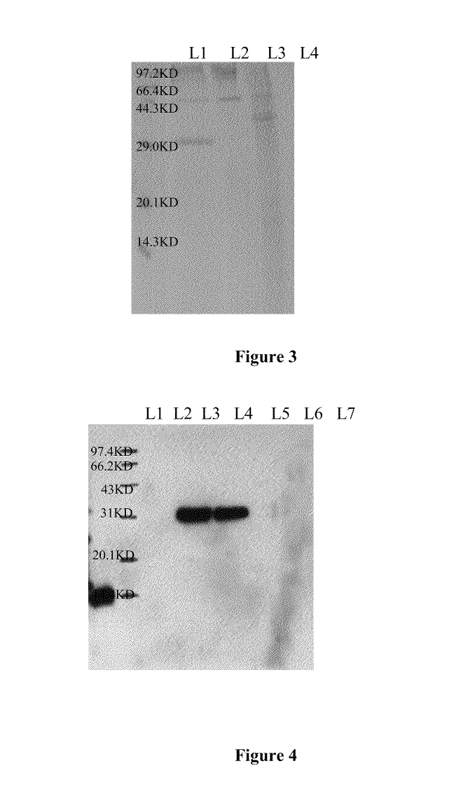

The expression of carboxylesterase is characterized using SDS-PAGE. Aspergillus niger M54 transformed with pYG1.2-CarE-his is cultured in uridine-free MN broth at 30° C. with 200 rpm shake for 5 days. Then the supernatants of the culture are taken and analyzed by electrophoresis. Supernatants of Aspergillus niger M54 transformed with pYG1.2 and supernatants of non-transformed Aspergillus niger M54 are analyzed in parallel as negative controls. 15 μl supernatants are mixed with 3 μl 6× loading SDS-PAGE buffer and boiled for 5 minutes before loading to the SDS-PAGE gel, which is composed of 12% polyacrylamide resolving gel and 5% polyacrylamide stacking gel. The samples are subjected to constant voltage electrophoresis at 120V for 3-4 hours. Then the gel is stained by Coomassie Brilliant Blue for 30 minutes at room temperature and rinsed to show the protein bands. A distinct protein band at 29.0 KD is observed in the lane of supernatants of Aspergillus niger M54 transformed with pYG1.2-CarE-his, but it is absent in the negative controls (FIG. 3).

The expression of carboxylesterase is further confirmed by Western Blot. Supernatants of Aspergillus niger M54 transformed with pYG1.2-CarE-his are separated by SDS-PAGE electrophoresis and proteins on the gel are transferred to polyvinylidene difluoride (PVDF) membrane for 1 hour by applying an electric current at an intensity of 0.65 mA/cm². The PVDF membrane is blocked with non-fat milk followed by incubation with an appropriate dilution of anti-his antibody (an IgG type antibody) at 4° C. overnight and incubation with a secondary antibody, an anti-IgG antibody, at room temperature for 90 minutes. After washing with PBST, the PVDF membrane is exposed to an X-ray film for an appropriate time period followed by visualization and photograph. Supernatants of non-transformed Aspergillus niger M54 and supernatants of Aspergillus niger M54 transformed with pYG1.2 vector are used as negative controls. His-tagged histones used as positive control. A distinct band at 29 KD is observed for the pYG1.2-CarE-his transformant while no band is detected in the negative controls (FIG. 4), confirming the expression of carboxylesterase in pYG1.2-CarE-his transformants.

### Example 2

### Expression of Carboxylesterase in Pichia pastoris GS115

The DNA sequence of the carboxylesterase contains an internal XhoI site CTCGAG, which is changed by a site specific silent mutation to CTCGAA (marked with double underline) using PCR. Two separate PCR reactions are performed using primers P3 and P4 shown in SEQ ID NOs: 36-37, and primers P5 and P6 shown in SEQ ID NOs:38-39, respectively, in which P3 contains an Xho I site, a KEX2 site and a KEX1 site, P4 and P5 contain the silent mutation, and P6 contains an EcoRI site and six histidine codons for a His-tag (coding sequence of His-tag is marked with a wave underline). The PCR conditions are the same as described in Example 1. The PCR products from the two separate reactions are purified by gel extraction after electrophoresis in 1% agarose gel. The PCR products from the two reactions have overlapping sequences (shown as underlined parts in P4 and P5 in Table 5) that would allow the two products to bind to each other at the 5′ end of one product and the 3′ end of the other product. Therefore, the two purified PCR products are mixed together for a third round of PCR to make a PCR product combining the two templates. Products of the third round of PCR are subjected to electrophoresis in 1% agarose gel and the band of about 800 bp is cut and purified by gel extraction.

TABLE 5
Nucleotide acid sequences of primers P3,
P4, P5 and P6, and the CarE-hisgene.

| Name | Sequence |
|---|---|
| P3 | 5′CGCTCGAGAAAAGAGAGGCTGAAGCTATGATGAAA |
| | ATTGTTCCG 3′ |
| | (SEQ ID NO: 36) |
| P4 | 5′CGCGCGCATATTCGAGGACGCCTTCGTAC 3′ |
| | (SEQ ID NO: 37) |
| P5 | 5′ CGTCCTCGAATATGCGCGCGAGTATAAAA 3′ |
| | (SEQ ID NO: 38) |
| P6 | 5′CGGAATTCTTA [symbol] CCAATC |
| | TAACGATTCAAG 3′ |
| | (SEQ ID NO: 39) |
| Mutated | 5′ATGATGAAAATTGTTCCGCCGAAGCCGTTTTTCTT |
| CarE-his | TGAAGCCGGGGAGCGGGCGGTGCTGCTTTTGCATGGG |
| | TTTACCGGCAATTCCGCCGACGTTCGGATGCTTGGGC |
| | GATTCTTGGAATCGAAAGGGTATACGTGCCACGCTCC |
| | GATTTACAAAGGGCATGGCGTGCCGCCGGAAGAGCTC |
| | GTCCACACCGGACCGGATGATTGGTGGCAAGACGTCA |
| | TGAACGGCTATCAGTTTTTGAAAAACAAAGGCTACGA |
| | AAAAATTGCCGTGGCTGGATTGTCGCTTGGAGGCGTA |
| | TTTTCTCTCAAATTAGGCTACACTGTACCTACACAAG |
| | GCATTGTGACGATGTGCGCGCCGATGTACATCAAAAG |
| | CGAAGAAACGATGTACGAAGGCGTCCTCGAATATGCG |
| | CGCGAGTATAAAAAGCGGGAAGGGAAATCAGAGGAAC |
| | AAATCGAACAGGAAATGGAACGGTTCAAACAAACGCC |
| | GATGAAGACGTTGAAAGCCTTGCAAGAACTCATTGCC |
| | GATGTGCGCGCCCACCTTGATTTGGTTTATGCACCGA |
| | CGTTCGTCGTCCAAGCGCGCCATGATGAGATGATCAA |
| | TCCAGACAGCGCGAACATCATTTATAACGAAATTGAA |
| | TCGCCGGTCAAACAAATCAAATGGTATGAGCAATCAG |
| | GCCATGTGATTACGCTTGATCAAGAAAAAGATCAGCT |
| | GCATGAAGATATTTATGCATTTCTTGAATCGTTAGAT |
| | TGG [symbol] TAA 3′ |
| | (SEQ ID NO: 40) |

The purified final PCR product and the pBluescriptII-SKM (Stratagene, La Jolla, Calif.) are separately digested using both XhoI and EcoRI, followed by electrophoresis in 1% agarose gel and purification by gel extraction. pBluescriptII-SKM contains a beta-lactamase gene that confers resistance to Ampicillin. After ligation of the digested PCR product and the digested pBluescriptII-SKM fragment, E. Coli DH5α are transformed with the ligation product following the same procedure described in Example 1. Individual colonies of E. Coli grown on the LB plates containing Ampicillin are screened using PCR, and positive colonies identified by PCR are propagated followed by plasmid purification. Target gene insertion is confirmed by digesting the purified plasmid with XhoI and EcoRI, followed by electrophoresis. The obtained recombinant plasmid is named as pBluescriptII-SKM-CarE-his and used in the following molecular cloning experiments.

Plasmid pBluescriptII-SKM-CarE-his is digested with XhoI and EcoRI, and ligated to the fragment of pPIC9 plasmid (Invitrogen) digested with the same restriction enzymes. The ligation product is used to transform E. Coli DH5α to obtain positive recombinant colonies identified by PCR and enzyme digestion with XhoI and EcoRI following the same procedures described in Example 1. The plasmid from the positive colonies is purified and named as pPIC9-CarE-his.

Plasmid pPIC9-CarE-his is digested with BamHI and EcoRI, and the fragment containing the carboxylesterase gene is ligated to the fragment of pPIC9K plasmid (Invitrogen) digested with the same restriction enzymes. pPIC9K plasmid carries a Kanamycin resistant gene which confers host cell resistance to Kanamycin as well as certain antibiotics that share structure similarity with Kanamycin. The ligation product is used to transform E. Coli DH5α to obtain positive recombinant colonies identified by PCR and enzyme digestion with BamHI and EcoRI following the same procedures described in Example 1. Electrophoresis results show a 1100 bp target band corresponding to the digestion fragment containing both the carboxylesterase gene and α-factor secreting signal sequence introduced from pPIC9 plasmid. The recombinant plasmid is named as pPIC9K-CarE-his and is used in subsequent studies.

pPIC9K-CarE-his plasmid is digested with BglII and then purified to obtain linearized plasmid DNA. 80 μl suspension of Pichia pastoris GS115 strain is mixed with the linearized plasmid DNA and equilibrated in ice cold cuvette for 5 minutes, followed by electroporation at 1500 v, 25 μF and 200Ω, and immediate addition of 1 ml ice cold 1M sorbitol. The mixture is placed on ice for 2-3 hours and then inoculated onto RDB plates. The plates are incubated at 30° C. for 3-5 days.

All colonies are washed from the RDB plates and diluted to about 10⁶ cells/ml with YPD medium. 100 μl diluted Pichia pastoris cells are inoculated onto YPD plates supplemented with various concentrations (0.25 mg/ml, 1 mg/ml and 2 mg/ml) of G418 (purchased from Invitrogen), which is an aminoglycoside antibiotic similar in structure to Kanamycin, followed by incubation at 30° C.

Individual colonies grown on the plates supplemented with the highest concentration of G418 are picked and inoculated separately into 3 ml MGY medium followed by incubation at 30° C. with shake at 250 rpm until the OD600 reaches 2-6. After centrifugation at 1500 g at room temperature for 5 minutes, the precipitates are resuspended with 3 ml BMMY medium (supplemented with 5‰ methanol) and incubated at 30° C. with shake at 250 rpm to induce expression of the target gene. Methanol (5‰) is added into the culture every 24 hours and samples of the culture are taken meanwhile. After 96 hours, the samples at different time intervals are run by SDS-PAGE analysis.

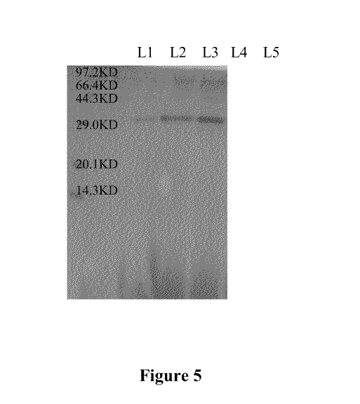

The supernatants of the samples are analyzed using SDS-PAGE electrophoresis and Western blot following the same procedures as described in Example 1. Supernatants of Pichia pastoris GS115 transformed with pPIC9K vector are analyzed in parallel as a negative control. Results obtained from electrophoresis and Western blot both show that a distinct protein band at 29.0 KD is present in the supernatants of Pichia pastoris GS115 transformed with pPIC9K-CarE-his but absent in the negative control (FIG. 4 and FIG. 5).

pPIC9K-CarE-his transformants expressing relatively higher level of carboxylesterase are picked, propagated and stored at −80° C.

### Example 3

### Characterization of Carboxylesterase Activity

Carboxylesterase activity is measured by incubating α-naphthyl acetate with supernatants of transformed Aspergillus niger M54 and supernatants of transformed Pichia pastoris GS115, respectively, at 37° C. pH 7.0 for 10 minutes followed by immediate termination of the reaction. Absorbance is measured at 600 nm. Enzyme activity units are calculated as the amount of enzyme needed to release 1 μmol α-naphthol from 0.03 M α-naphthyl acetate solution per minute.

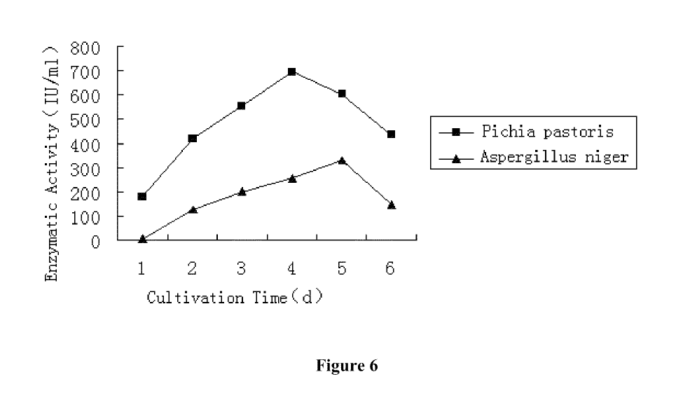

To determine the incubation time period for producing the highest enzyme production, the culture supernatants containing carboxylesterase are sampled on the 1st day, 2nd day, 3rd day, 4th day, 5th day and 6th day of the incubation, and then measured for carboxylesterase activity. As shown in FIG. 6, recombinant carboxylesterase activity reaches its peak in transformed Aspergillus niger M54 after 5-day incubation and in transformed Pichia pastoris GS115 after 4-day incubation, and begins to decline thereafter. Carboxylesterase is produced in an amount of 15.3 mg/ml in the 5th day culture of transformed Aspergillus niger M54, and is produced in an amount of 30.7 mg/ml in the 4th day culture of transformed Pichia pastoris GS115, as determined by gel imaging analysis system. The results suggest that the proper incubation period for carboxylesterase production in transformed Aspergillus niger M54 and transformed Pichia pastoris GS115 are 5 days and 4 days, respectively. The production yield of recombinant carboxylesterase in transformed Pichia pastoris GS115 is higher than that in transformed Aspergillus niger M54.

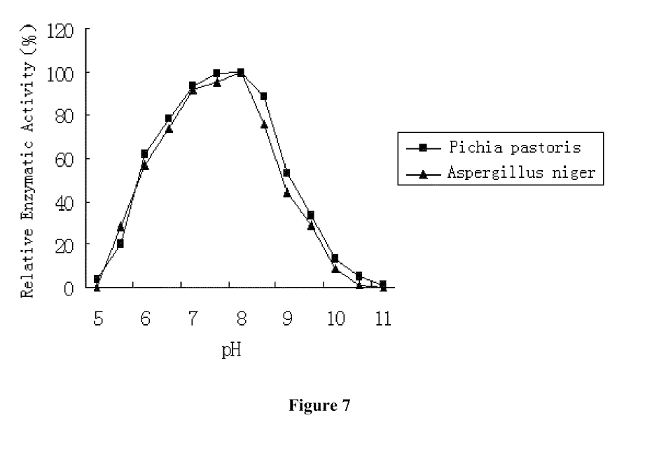

To test the pH dependence of the carboxylesterase activity, the culture supernatants containing carboxylesterases expressed from both transformed Aspergillus niger M54 and transformed Pichia pastoris GS115 are incubated with a substrate in buffers having pH values ranging from 5 to 11 at 37° C. for 30 minutes, followed by enzymatic reaction and activity determination. The results show that the recombinant carboxylesterase exhibits the highest enzymatic activity at pH 8.0, which is defined as 100% relative enzymatic activity, and above 75% relative enzymatic activity within the pH range of 6.0 and 8.5 and decreased activity when pH is below 6.0 or above 8.5 (FIG. 7).

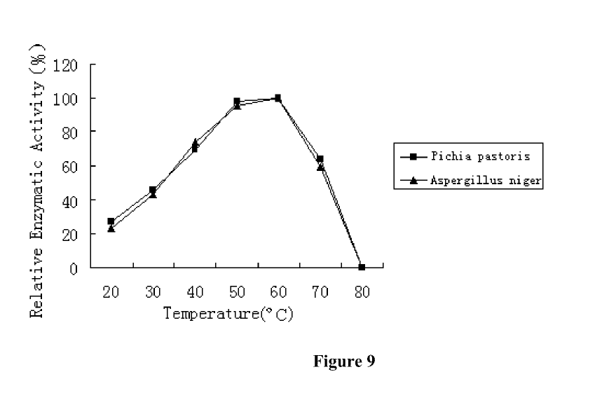

To determine the proper reaction temperature for carboxylesterase activity, the culture supernatants containing carboxylesterases are incubated with substrate solutions at temperatures ranging from 20° C. to 80° C. at pH 7.0 for 30 minutes followed by determination of enzymatic activities. Carboxylesterases expressed from both transformed Aspergillus niger M54 and transformed Pichia pastoris GS115 show highest enzymatic activity at 60° C., which is defined as 100% relative enzymatic activity, and decreased activity at higher temperatures at 70° C. and 80° C. (FIG. 9).

To test the thermostability of the recombinant carboxylesterase, the culture supernatants containing carboxylesterases expressed from both transformed Aspergillus niger M54 and transformed Pichia pastoris GS115 are incubated at temperatures ranging from 40° C. to 80° C. for 10 minutes and 30 minutes, respectively, followed by cooling in ice-bath. The substrate solutions are added to the enzyme incubations followed by enzymatic reaction at 37° C. for 10 minutes and activity determination. The results show that, after incubation at 60° C. for 10 minutes, the recombinant carboxylesterase retains nearly 100% of the enzymatic activity, and exhibits about 60% relative enzymatic activity after incubation at 70° C. for 30 minutes (FIG. 8).

### Example 4

### Expression of Carboxylesterase from Geobacillus kaustophilus HTA426 Strain in Hansenula polymorpha

The carboxylesterase from Geobacillus kaustophilus HTA426 strain (Accession number: BA000043, SEQ ID NO: 10) is cloned from the cDNA library of Geobacillus kaustophilus HTA426 strain by PCR using primers designed from the known DNA sequence. PCR products are purified and digested with appropriate restriction enzymes, and then ligated with pHIPX4 vector (Gietl et al, Mutational analysis of the N-terminal topogenic signal of watermelon glyoxysomal malate dehydrogenase using the heterologous host Hansenula polymorpha. Proc Natl Sci USA 1994, vol 91, 31513155). The recombinant pHIPX4 plasmid with insertion of the carboxylesterase gene is propagated in E. Coli DH5α and is then linearized to transform Hansenula polymorpha strain leu 1.1 (Gleeson et al, Transformation of the methylotrophic yeast Hansenula polymorpha. J Gen Microbiol 1986, vol, 132, 3459-65) using electroporation.

The transformed Hansenula polymorpha are screened using leucine-free culture medium. Single clones growing on leucine-free culture plates are incubated and the expression of carboxylesterase is characterized using enzymatic assays with α-naphthyl acetate as a substrate.

### Example 5

### Expression of Carboxylesterase from Bacillus thermoleovorans Strain in Aspergillus oryzae

The carboxylesterase from Bacillus thermoleovorans strain (Accession number: AF327065, SEQ ID NO:4) is cloned from the cDNA library of Bacillus thermoleovorans strain by PCR using primers designed from the known DNA sequence. PCR products are purified and digested with appropriate restriction enzymes, and then ligated with pSa123 vector which carries the Arginine synthesis gene (Gomi et al, Integrative transformation of Aspergillus oryzae with a plasmid containing the Aspergillus nidulans argB gene. Agric. Biol. Chem. 1987, vol 51, 2549-2555). The recombinant pSa123 plasmid with insertion of the carboxylesterase gene is propagated in E. Coli DH5α and is then linearized to transform Aspergillus oryzae M-2-3 which is deficient in the Arginine synthesis gene (Ozeki et al, Construction of a promoter probe vector autonomously maintained in Aspergillus and characterization of promoter regions derived from A. niger and A. oryzae genomes. Biosci. Biotech. Biochem. 1996, vol 60, 383-389) using Aspergillus oryzae M-2-3 protoplast.

The transformed Aspergillus oryzae are screened using arginine-free culture medium. Single clones growing on arginine-free culture plates are incubated and the expression of carboxylesterase is characterized using enzymatic assays with α-naphthyl acetate as substrate.

### Example 6

### Purification of the Expressed Carboxylesterase

Pichia pastoris GS115 transformed with pPIC9K-CarE-his are cultured at 30° C. for 4 days. The culture medium is harvested and filtered through a 0.2 μm filter followed by addition of NaAzide to a final concentration of 0.01%. 100 ml glycerol, 30 ml 5 M NaCl, 10 ml 1M imidazole and 50 ml Ni-NTA superflow resin are added to each liter of the harvested culture medium, followed by Gyro-rotary motion at 150 rpm at room temperature for 30-40 minutes. The resin beads are spin down at 3750 rpm for 10 minutes. The beads are loaded to a column and the column is washed with washing buffer containing: 50 mM Tris (pH 8.0), 300 mM NaCl, 10% Glycerol, and 10 mM Imidazole, until UV absorbance at 280 nm is stable. Then the column is eluted with elution buffer containing: 50 mM Tris pH 8.0, 300 mM NaCl, 10% Glycerol, and 250 mM Imidazole, until no protein is detected in the eluted solution. The eluted fractions are analyzed by electrophoresis to identify fractions with a single target protein band. Target fractions are collected and tested for the quantity and enzymatic activity.

### General

With respect to the use of substantially any plural and/or singular terms herein, those having skill in the art can translate from the plural to the singular and/or from the singular to the plural as is appropriate to the context and/or application.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells and so forth.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method for producing a protein, comprising:
culturing a eukaryotic cell engineered to express a gene encoding a thermostable carboxylesterase from a microbe or its variant under conditions suitable for expression of the thermostable carboxylesterase or its variant.

2. The method of claim 1, wherein the eukaryotic cell is a yeast cell.

3. The method of claim 2, wherein the yeast cell is selected from the group consisting of Pichia species, Hansenula species, Saccharomyces species and Candida species.

4. The method of claim 3, wherein the yeast cell is selected from the group consisting of Pichia pastoris, Hansenula polymorpha, Saccharomyces cerevisiae and Torulopsis glabrata.

5. The method of claim 4, wherein the yeast cell is Pichia pastoris GS115.

6. The method of claim 1, wherein the eukaryotic cell is a filamentous fungal cell.

7. The method of claim 6, wherein the filamentous fungal cell is from the Aspergillus genus.

8. The method of claim 7, wherein the filamentous fungal cell is selected from the group consisting of Aspergillus niger and Aspergillus oryzae.

9. The method of claim 8, wherein the filamentous fungal cell is Aspergillus niger M54.

10. The method of claim 1, further comprising isolating the carboxylesterase or its variant from the eukaryotic cell culture.

11. The method of claim 1, further comprising introducing an expression vector containing the gene encoding for the carboxylesterase or its variant into the eukaryotic cell.

12. The method of claim 1, wherein the expressed carboxylesterase or its variant is secreted to the outside of the eukaryotic cell.

13. The method of claim 1, wherein the carboxylesterase is a bacterial carboxylesterase.

14. (canceled)

15. The method of claim 1, wherein the carboxylesterase or its variant has at least 70% sequence identity to the amino acid sequence of a carboxylesterase isolated from Geobacillus stearothermophilus.

16. The method of claim 15, wherein the carboxylesterase isolated from Geobacillus stearothermophilus has the amino acid sequence as set forth in SEQ ID NOs: 9, 11, 13 or 15.

17. The method of claim 16, wherein the carboxylesterase or its variant has at least 90% sequence identity to the amino acid sequence encoded by any of SEQ ID NOs: 10, 12, 14 or 16.

18-31. (canceled)

32. A recombinant eukaryotic cell comprising an expression vector comprising a gene encoding a microbial thermostable carboxylesterase or its variant; and a regulatory sequence capable of promoting expression of the microbial thermostable carboxylesterase or its variant in a eukaryotic cell, wherein the regulatory sequence is operably linked to the gene.

33. The eukaryotic cell of claim 32, wherein the cell is Aspergillus niger.

34. The eukaryotic cell of claim 32, wherein the cell is Pichia pastoris.

35-37. (canceled)


SEQUENCE LISTING (40 sequences)

SEQ ID NO: 1 - protein, 246 aa, Geobacillus kaustophilus
MKIVPPKPFFFEAGERAVLLLHGFTGNSADVRMLGRFLESKGYTCHAPIYKGHGVPPEELVHTGPDDWWQDVMNGYQFLKNKGYEKIAVAGLSLGGVFSLKLGYTVPIQGIVTMCAPMYIKSEETMYEGVLEYAREYKKREGKSEEQIEQEMERFKQTPMKTLKALQELIADVRAHLDLVYAPTFVVQARHDEMINPDSANIIYNEIESPVKQIKWYEQSGHVITLDQEKDQLHEDIYAFLESLDW

SEQ ID NO: 2 - DNA, 741 bp, Geobacillus kaustophilus
ttaccaatctaacgattcaagaaatgcataaatatcttcatgcagctgatctttttcttgatcaagcgtaatcacatggcctgattgctcataccatttgatttgtttgaccggcgattcaatttcgttataaatgatgttcgcgctgtctggattgatcatctcatcatggcgcgcttggacgacgaacgtcggtgcataaaccaaatcaaggtgggcgcgcacatcggcaatgagttcttgcaaggctttcaacgtcttcatcggcgtttgtttgaaccgttccatttcctgttcgatttgttcctctgatttcccttcccgctttttatactcgcgcgcatactcgagcacaccttcgtacatcgtttcttcgcttttgatgtacatcggcgcgcacatcgtcacaatgccttgtataggtacagtgtagcctaatttgagagaaaatacgcctccaagcgacaatccagccacggcaattttttcgtagcctttgtttttcaaaaactgatagccgttcatgacgtcttgccaccaatcatccggtccggtgtggacgagctcttccggcggcacgccatgccctttgtaaatcggagcgtggcacgtataccctttcgattccaagaatcgcccaagcatccgaacgtcggcggaattgccggtaaacccatgcaaaagcagcaccgcccgctccccggcttcaaagaaaaacggcttcggcggaacaattttcat

SEQ ID NO: 3 - protein, 246 aa, Geobacillus thermoleovorans
MMKIVPPKPFFFEAGERAVLLLHGFTGNSADVRMLGRFLESKGYTCHAPITKGMVPPEELVHTGPDDWWQDVMNGYQFLKNKGYEKIAVAGLSLGGVFSLKLGYTVPIEGIVTMCAPMYIKSEETMYEGVLEYAREYKKREGKSEEQIEQEMERFKQTPMKTLKALQELIADVRAHLDLVYARTFVVQARHDKMINPDSANIIYNEIESPVKQIKWYEQSGHVITLDQEKDQLHEDIYAFLESLDW

SEQ ID NO: 4 - DNA, 741 bp, Geobacillus thermoleovorans
atgatgaaaattgttccgccgaagccgtttttctttgaagccggggagcgggcggtgctgcttttgcatgggtttaccggcaattccgccgacgttcggatgcttgggcgattcttggaatcgaaagggtatacgtgccacgctccgattacaaagggcatggtgccgccggaagagctcgtccacaccggaccggatgattggtggcaagacgtcatgaacggctatcagtttttgaaaaacaaaggctacgaaaaaattgccgtggctggattgtctcttggaggcgtattttctctcaaattaggctacactgtacctatagaaggcattgtgacgatgtgcgcgccgatgtacatcaaaagcgaagaaacgatgtacgaaggtgtgctcgagtatgcgcgcgagtataaaaagcgggaagggaaatcagaggaacaaatcgaacaggaaatggaacggttcaaacaaacgccgatgaagacgttgaaagccttgcaagaactcattgccgatgtgcgcgcccaccttgatttggtttatgcacgcacgttcgtcgtccaagcgcgccatgataagatgatcaatccagacagcgcgaacatcatttataacgaaattgaatcgccggtcaaacaaatcaaatggtatgagcaatcaggccatgtgattacgcttgatcaagaaaaagatcagctgcatgaagatatttatgcatttcttgaatcgttagattggtaa

SEQ ID NO: 5 - protein, 256 aa, Salmonella enterica
MNDIWWQTYGEGNCHLVLLHGWGLNAEVWHCIREELGSHFTLHLVDLPGYGRSSGFGAMTLEEMTAQVAKNAPDQAIWLGWSLGGLVASQMALTHPERVQALVTVASSPCFSAREGWPGIKPEILGGFQQQLSDDFQRTVERFLALQTLGTETARQDARTLKSVVLAQPMPDVEVLNGGLEILKTVDLREALKNVNMPFLRLYGYLDGLVPRKIAPLLDTLWPHSTSQIMAKAAHAPFISHPAAFCQALMTLKSSL

SEQ ID NO: 6 - DNA, 771 bp, Salmonella enterica
ttacagcgatgattttagcgtcatcagcgcctgacaaaacgccgccggatgcgagataaacggcgcatgggccgccttcgccattatctgtgatgtactgtgcggccataacgtatcgagcaaaggcgcgattttacgcggcaccagaccgtccagataaccatacaaacgcaaaaacggcatgttcacatttttaagcgcttctcgtagatcgaccgtttttaagatttccagtccgccattgagcacctctacatccggcataggctgcgccagcactacgctttttaaggtgcgggcatcctgacgcgccgtctccgtccctaacgtttgcagcgccagaaaacgctccaccgtgcgctgaaaatcgtcgctaagctgctgctggaatccgccgaggatttctggttttattcccggccacccctcacgcgcgctaaagcatggcgaagaggcgactgtcaccagcgcctgaacgcgttcagggtgggtgagcgccatctgactcgccaccaggccgcccaggctccagccaagccagatagcctggtccggcgcgtttttcgctacctgcgccgtcatctcttcaagcgtcatggcgccaaaccccgagctgcgaccatagcccggcaagtcgaccagatgcagcgtaaaatgcgagccgagttcctcgcgaatgcaatgccatacctccgcgttcaatccccatccgtgcagcagcacaagatgacaatttccctcgccgtaggtctgccaccagatgtcattcat

SEQ ID NO: 7 - protein, 541 aa, Aspergillus fumigatus
MVISTKYIFALCVLLLTFSLSSAYEDPLVELDYGQFQGKYDSTYNLSYFRKIPFAAPPTGENRFRAPQPPLRITHGVYDTDQDFDMCPQRTVNGSEDCLYLGLFSRPWDATVAPSSASRPVLVVFYGGGFIQGSASFTLPPSSYPILNVTELNDYVVIYPNYRVNAFGFLPGKAIKRSPTSDLNPGLLDQQYVLKWVQKYIHHFGGDPRNVTIWGQSAGAGSVVAQVLANGRGNQPKLFDKALVSSPFWPKTYAYDAPEAEAIYDQLVTLTGCANATDSLACLKSVDVQTIRDANLIISASHTYNTSSYTWAPVIDGEFLQESLTTAVARRKIKTHFVFGMYNTHEGENFLPSGLGKTATTAGFNSSAASFHTWLTGFLPGLSPKHIALLETKYYPPTGETETIDLYNTTLVRAGLVYRDLVLACPAYWLTSAARRRGYLGEYTISPAKHASDTIYWNRVNPIQQTDPLIYDGFAGAFGSFFQTGDPNAHKLTNASEKGVPVLEKTGEEWVIAPDGFATAQVSFLKERCDFWESVGERVPV

SEQ ID NO: 8 - DNA, 1626 bp, Aspergillus fumigatus
atggtgatctcaacgaagtatatatttgccctttgcgtcctcttgctgaccttctcactctctagcgcctacgaagatcccctcgtcgagctcgactatgggcagttccagggcaaatatgactctacgtataatctctcatacttccgcaagatcccctttgcggcgcctccaacgggggagaaccggtttagagcccctcagccacctctaaggatcacgcatggcgtctatgacactgatcaggactttgacatgtgcccgcaacgcacggtcaatggctccgaagactgcctctaccttggcctgttctcacgaccgtgggatgctacagtggctccctcgtctgcttctagaccagtcctggtagtcttctacggtggtggcttcatccaaggcagcgcttcgtttacactacctccgtcctcatatccaatcctgaacgtcaccgagctgaatgactatgtggtcatctaccccaactaccgggtcaatgcatttggtttccttccgggcaaggcgatcaagcgatctccaacgtctgatctcaaccccggcctcttggaccagcagtacgttctcaagtgggtgcagaaatacattcaccacttcggcggtgaccctcgcaacgtcacgatctggggccaatccgccggcgccggctcagtggttgcgcaggttctcgccaacggacgaggcaaccaacccaagctcttcgacaaagcgctcgtcagctcgcccttctggccaaagacctacgcctacgacgcccccgaagcagaagccatctacgaccagcttgtcactctcaccggctgcgccaatgccaccgactccctcgcctgcctgaaatccgtcgacgtccagaccatccgcgacgcaaacctcatcatcagcgccagccacacctacaacacaagctcctacacctgggcccccgtcatcgacggcgaattcctccaagaatccctcaccaccgccgtcgcccgccgcaaaatcaaaacccacttcgtcttcggcatgtacaacacccacgagggcgagaacttcctcccctccggcctgggcaagaccgctacaaccgctgggttcaactcctctgctgctagcttccacacctggctgacgggcttcctgccgggtctctcgcccaagcacatcgccctcctcgaaaccaagtactacccgcccaccggcgaaacagagacaatcgacctgtacaacacgacgctcgtccgcgcgggcctggtctacagggatcttgtcctcgcctgtccggcgtactggcttacctcggccgcaagacggaggggctatctaggtgaatatacgatttcgccggctaagcacgcgagcgataccatctattggaaccgagtgaacccgatccagcagactgatccactgatttatgacggcttcgcaggcgcttttgggagtttcttccagacgggcgatccgaatgcgcataagctgacgaatgcgtcggagaagggtgtgccggttctcgagaagaccggggaggagtgggtgattgctccggatgggtttgcaaccgcgcaggtgtcgtttttaaaggagaggtgtgatttctgggagtcggtgggggagcgggttcctgtctga

SEQ ID NO: 9 - protein, 247 aa, Geobacillus stearothermophilus
MMKIVPPKPFFFEAGERAVLLLHGFTGNSADVRMLGRFLESKGYTCHAPIYKGHGVPPEELVHTGPDDWWQDVMNGYEFLKNKGYEKIAVAGLSLGGVFSLKLGYTVPIEGIVTMCAPMYIKSEETMYEGVLEYAREYKKREGKSEEQIEQEMEKFKQTPMKTLKALQELIADVRDHLDLIYAPTFVVQARHDEMINPDSANIIYNEIESPVKQIKWYEQSGHVITLDQEKDQLHEDIYAFLESLDW

SEQ ID NO: 10 - DNA, 744 bp, Geobacillus stearothermophilus
atgatgaaaattgttccgccgaagccgtttttctttgaagccggggagcgggcggtgctgctgttgcatgggtttaccggcaattccgctgatgttcggatgctcgggcgttttttagaatccaaaggctatacgtgccatgcgcctatttacaaaggacacggcgtgccgcctgaggagctcgtccacaccgggccggatgactggtggcaagatgtcatgaacggctacgagtttttgaaaaacaagggctacgaaaaaatcgccgtcgccggactgtcgcttggaggcgtattttcattgaaattaggttacactgtacctatagagggcattgtgacgatgtgcgcgccgatgtacatcaaaagcgaggaaacgatgtacgaaggcgtgctcgagtatgcgcgcgagtataaaaagcgggaaggaaaatcagaggagcagatcgaacaggagatggagaagttcaagcagacgccgatgaagacgttaaaggcgctgcaggagctgatcgccgatgtgcgtgaccatcttgatttgatttatgccccgacgtttgttgtgcaggcgcgccatgatgagatgatcaacccggacagcgcgaacatcatttataacgaaattgaatcgccggtcaaacaaatcaagtggtatgagcaatcaggccatgtgattacgcttgatcaagaaaaagatcagctgcatgaagatatttatgcatttcttgaatcgttagattggtaa

SEQ ID NO: 11 - protein, 176 aa, Geobacillus stearothermophilus
KLGEKELLDRINREVGPVPEEAIRYYKETAEPSAPTWQTWLRIMTYRVFVEGMLRTADAQAAQGADVYMYRFDYETPVFGGQLKACHALELPFVFHNLHQPGVANFVGNRPEREAIANEMHYAWLSFARTGDPNGAHLPEAWPAYTNERKAAFVFSAASHVEDDPFGRERAAWQGR

SEQ ID NO: 12 - DNA, 531 bp, Geobacillus stearothermophilus
aagcttggcgaaaaggaacttcttgaccgaatcaaccgggaagtcgggccggtgccagaagaggccatccgctattacaaagaaacggcggagccgtcggcgcctacttggcaaacgtggcttcgcatcatgacgtaccgcgtatttgtcgaggggatgctgcggacggcggacgcccaagcggcgcaaggggcggatgtgtacatgtaccgctttgactatgagacgccggtgttcggcggccagctgaaagcatgccacgcgctcgagctgccgtttgtgtttcacaatctccatcagccgggcgtcgcgaatttcgtcggcaaccggccggagcgcgaggcgatcgccaatgaaatgcattacgcttggctctcgtttgcccgcaccggagacccgaacggtgctcacttgccggaagcgtggccggcgtatacgaacgagcgcaaggcggcctttgtcttttcggccgccagccatgtcgaggacgacccgttcggccgcgagcgggcggcatggcaaggacgctag

SEQ ID NO: 13 - protein, 498 aa, Geobacillus stearothermophilus
MERTVVETRYGRLRGEMNEGVFVWKGIPYAKAPVGERRFLPPEPPDAWDGVREATSFGPVVMQPSDPIFSGLLGRMSEAPSEDGLYLNIWSPAADGKKRPVLFWIHGGAFLFGSGSSPWYDGTAFAKHGDVVVVTINYRMNVFGFLHLGDSFGEAYAQAGNLGILDQVAALRWVKENIAAFGGDPDNITIFGESAGAASVGVLLSLPEASGLFRRAMLQSGSGSLLLRSPETAMAMTERILDKAGIRPGDRERLLSIPAEELLRAALSLGPGVMYGPVVDGRVLRRHPIEALRYGAASGIPILIGVTKDEYNLFTLTDPSWTKLGEKELLDRINREVGPVPEEAIRYYKETAEPSAPTWQTWLRIMTYRVFVEGMLRTADAQAAQGADVYMYRFDYETPVFGGQLKACHALELPFVFHNLHQPGVANFVGNRPEREAIANEMHYAWLSFARTGDPNGAHLPEAWPAYTNERKAAFVFSAASHVEDDPFGRERAAWQGR

SEQ ID NO: 14 - DNA, 1497 bp, Geobacillus stearothermophilus
atggagcgaaccgttgttgaaacaaggtacggacggttgcgcggggaaatgaatgaaggcgtttttgtttggaaaggaattccgtacgcgaaagcgccggtcggtgagcgccggtttttgccgccggagccgcccgatgcgtgggatggggtgcgggaggcgacatcgttcggtcctgtcgtgatgcagccgtcggatccgattttcagcggattgctcgggcggatgagcgaggcgccgagcgaagacgggctgtacttgaacatttggtcgccggcggcggatgggaagaagcgcccggtgttgttttggattcacggcggcgcctttttgttcggttcgggttcttcgccgtggtatgacgggacggcgttcgcgaaacacggcgatgtcgttgtcgtgacgatcaactaccgaatgaacgtgtttggctttttgcatctcggtgattcgttcggcgaagcgtacgcgcaagccgggaatctcggcattttggaccaagtggcggcgctgcgctgggtgaaggagaacattgcggcgtttggtggtgatccggacaacatcacgattttcggtgaatcggccggagcggcgagcgtcggcgtgctgttgtcgcttccggaggccagcgggctgtttcggcgcgccatgttgcaaagcggttcgggatcgcttcttctccgttcgccggagaccgcgatggcgatgaccgaacgcattcttgataaggctggcatccgtccgggcgaccgcgaacggctgttgtcgattcctgccgaggagctgctgcgggcggcgctgtcgctcggtccaggggtcatgtacggtccggtggtggatggccgcgtattgcgccgccatccgatcgaagcgctccgctacggggcggccagcggcattccgattctcatcggcgtgacgaaagacgagtacaacttgtttaccttgacggatccgtcatggacaaagcttggcgaaaaggaacttcttgaccgaatcaaccgggaagtcgggccggtgccagaagaggccatccgctattacaaagaaacggcggagccgtcggcgcctacttggcaaacgtggcttcgcatcatgacgtaccgcgtatttgtcgaggggatgctgcggacggcggacgcccaagcggcgcaaggggcggatgtgtacatgtaccgctttgactatgagacgccggtgttcggcggccagctgaaagcatgccacgcgctcgagctgccgtttgtgtttcacaatctccatcagccgggcgtcgcgaatttcgtcggcaaccggccggagcgcgaggcgatcgccaatgaaatgcattacgcttggctctcgtttgcccgcaccggagacccgaacggtgctcacttgccggaagcgtggccggcgtatacgaacgagcgcaaggcggcctttgtcttttcggccgccagccatgtcgaggacgacccgttcggccgcgagcgggcggcatggcaaggacgctag

SEQ ID NO: 15 - protein, 226 aa, Geobacillus stearothermophilus
MHNDLAYEYDIHLPSGGEAGKKYPAVFALHGIGYDEQYMLTLVKDLKEEFILIGIRGDLPYEDGYAYYYLKEYGKPERKMFDDSIGKLKHFIEYALNQYPIDSDRVYLIGFSQGAILSMSLALILGDKIKGIAAMNGYIPSFVKEEYPLQPISHLSVFLTQGESDHIFPLNIGQENYEYLRQHAGAVKYTIYPAGHEISQDNQRDIVSWLRHDAFHHNSNKATNPA

SEQ ID NO: 16 - DNA, 681 bp, Geobacillus stearothermophilus
atgcataatgacttggcatatgaatatgacattcatcttccttctggcggagaagcggggaaaaagtatccggctgttttcgcgttgcacggcatcgggtatgacgaacaatacatgcttactttagtgaaagatttaaaagaagaatttattttaataggcattagaggggatctcccgtatgaagatggatatgcctattattatttgaaagaatatggaaagccagaacggaaaatgttcgatgatagcataggcaaattaaagcacttcattgaatatgcattaaaccaatatccgattgattccgatcgagtgtatttgatcgggtttagtcaaggcgccattttaagtatgtctctcgccttgatactgggcgataaaattaaagggattgccgcaatgaacggatatataccatcgtttgtgaaggaagaatatccgttgcagcctatcagtcacttgtctgtgtttctcacccaaggcgaatcagatcatatttttcctttaaatattgggcaggaaaattatgaatacttgcgccagcatgcgggggctgtgaagtataccatttatccggcaggacatgaaatatcgcaagacaaccaacgcgacatcgtttcatggctgcgtcatgatgcattccatcacaattccaataaggcaacaaatcccgcatga

SEQ ID NO: 17 - protein, 454 aa, Homo sapiens
EHCLYLNIYTPADLTKKNRLPVMVWIHGGGLMVGAASTYDGLALAAHENVVVVTIQYRLGIWGFFSTGDEHSRGNWGHLDQVAALRWVQDNIASFGGNPGSVTIFGESAGGESVSVLVLSPLAKNLFHRAISESGVALTSVLVKKGDVKPLAEQIAITAGCKTTTSAAMVHCLRQKTEEELLETTLKIGNSYLWTYRETQRESTLLGTVIDGMLLLKTPEELQRERNFHTVPYMVGINKQEFGWLIPMQLMSYPLSEGQLDQKTAMSLLGSPIPLFAIAKELIPEATEKYLGGTDDTVKKKDLILDLIADVMFGVPSVIVARNHRDAGAPTYMYEFQYRPSFSSDMKPKTVIGDHGDELFSVFGAPFLKEGASEEEIRLSKMVMKFWANFARNGNPNGKGLPHWPEYNQKEGYLQIGANTQAAQKLKDKEVAFWTNLFAKKAVEKPPQTDHIEL

SEQ ID NO: 18 - DNA, 1367 bp, Homo sapiens
ctgaacactgtctttacctcaatatttacactcctgcagacttgaccaagaaaaacaggctgccggtgatggtgtggatccacggaggggggctgatggtgggtgcggcatcaacctatgatgggctggcccttgctgcccatgaaaacgtggtggtggtgaccattcaatatcgcctgggcatctggggattcttcagcacaggggatgaacacagccgggggaactggggtcacctggaccaggtggctgccctgcgctgggtccaggacaacattgccagctttggagggaacccaggctctgtgaccatctttggagagtcagcgggaggagaaagtgtctctgttcttgttttgtctccattggccaagaacctcttccaccgggccatttctgagagtggcgtggccctcacttctgttctggtgaagaaaggtgatgtcaagcccttggctgagcaaattgctatcactgctgggtgcaaaaccaccacctctgctgctatggttcactgcctgcgacagaagacggaagaggagctcttggagacgacattgaaaattggaaattcttatctctggacttacagggagacccagagagagtcaacccttctgggcactgtgattgatgggatgctgctgctgaaaacacctgaagagcttcaacgtgaaaggaatttccacactgtcccctacatggtcggaattaacaagcaggagtttggctggttgattccaatgcagttgatgagctatccactctccgaagggcaactggaccagaagacagccatgtcactccttggaagtcctatccccttgtttgccattgctaaggaactgattccagaagccactgagaaatacttaggaggaacagacgacactgtcaaaaagaaagacctgatcctggacttgatagcagatgtgatgtttggtgtcccatctgtgattgtggcccggaaccacagagatgctggagcacccacctacatgtatgagtttcagtaccgtccaagcttctcatcagacatgaaacccaagacggtgataggagaccacggggatgagctcttctccgtctttggggccccatttttaaaagagggtgcctcagaagaggagatcagacttagcaagatggtgatgaaattctgggccaactttgctcgcaatggaaaccccaatgggaaagggctgccccactggccagagtacaaccagaaggaagggtatctgcagattggtgccaacacccaggcggcccagaagctgaaggacaaagaagtagctttctggaccaacctctttgccaagaaggcagtggagaagccaccccagacagaccacatagagctgtga

SEQ ID NO: 19 - protein, 565 aa, Mus musculus
MWLCALSLISLTACLSLGHPSLPPVVHTVHGKVLGKYVTLEGFSQPVAVFLGVPFAKPPLGSLRFAPPEPAEPWSFVKHTTSYPPLCYQNPEAALRLAERFTNQRKIIPHKFSEDCLYLNIYTPADLTQNSRLPVMVWIHGGGLVIDGASTYDGVPLAVHENVVVVVIQYRLGIWGFFSTEDEHSRGNWGHLDQVAALHWVQDNIANFGGNPGSVTIFGESAGGESVSVLVLSPLAKNLFHRAIAQSSVIFNPCLFGRAARPLAKKIAALAGCKTTTSAAMVHCLRQKTEDELLEVSLKMKFGTVDFLGDPRESYPFLPTVIDGVLLPKAPEEILAEKSFNTVPYMVGINKHEFGWIIPMFLDFPLSERKLEQKTAASILWQAYPILNISEKLIPAAIEKYLGGTEDPATMTDLFLDLIGDIMFGVPSVIVSRSHRDAGAPTYMYEYQYRPSFVSDDRPQELLGDHADELFSVWGAPFLKEGASEEEINLSNMVMKFWANFARNGNPNGEGLPHWPEYDQKEGYLQIGVPAQAAHRLKDKEVDFWTELRAKETAERSSHREHVEL

SEQ ID NO: 20 - DNA, 1698 bp, Mus musculus
atgtggctctgtgctttgagtctgatctctctcactgcttgcttgagtctgggacacccatccttaccgcctgtggtacacaccgttcatggcaaagtcctggggaagtatgtcaccttagaaggattctcacagcctgtggccgtcttcctgggagtcccctttgccaagccccctcttggatctctgaggtttgctccaccagagcctgcagagccctggagcttcgtgaagcacaccacttcctaccctcctttgtgctaccaaaacccagaggcagcattgaggctcgctgagcgcttcaccaaccaaaggaagatcattccccacaaattttctgaggactgtctctacctcaacatttatactcctgctgacttaacacagaacagcaggttgcccgtgatggtgtggatacatggaggtggacttgtgatagatggagcatcaacctatgatggagtgcccctggctgtccatgaaaatgtggttgtagtggtcattcagtatcgcctgggcatctggggattcttcagcacagaggatgaacacagccgggggaactggggtcacttggaccaggtggctgcactacattgggtccaagacaacattgccaactttgggggcaacccaggatctgtgactatcttcggcgagtcagcaggaggtgaaagtgtctctgttcttgtgttaagcccactggccaagaacctcttccacagggccatcgctcagagtagtgtcattttcaatccttgcctttttgggagagctgccagacccttggctaagaaaattgctgctcttgctggctgtaaaaccaccacctccgctgccatggttcactgcctgcgccagaagactgaagatgagctcttggaggtctcactgaaaatgaaatttgggactgttgattttcttggagaccccagagagagctatcccttcctccctactgtgattgatggagtgttgctgccaaaggcaccagaagagattctggctgagaagagtttcaacactgtcccctacatggtgggcatcaacaagcatgagtttggctggatcattccaatgtttttggacttcccactctctgaaagaaaactggaacagaagacagctgcatccatcctgtggcaggcctacccaattcttaacatctctgaaaagctgattccagcagctattgaaaagtatttaggagggacagaagaccctgccacaatgacagacctgttcctggacttgattggagacattatgttcggtgtcccatctgtaatcgtgtcccgtagtcacagagatgctggagccccaacctacatgtatgaatatcagtatcgcccaagttttgtatcagacgatagaccccaggaattgttaggagaccacgctgatgaactcttttctgtatggggagccccgtttttaaaagagggtgcttcagaagaagagatcaacctcagcaacatggtgatgaaattctgggccaactttgctcggaatgggaaccctaatggtgaagggctgcctcattggccagaatatgaccagaaggaaggataccttcagattggagtcccagcacaggcagcccataggctgaaagacaaggaagtggacttttggactgagctcagagccaaggaaacagcagagaggtcatcccatagggaacatgttgaactgtga

SEQ ID NO: 21 - protein, 553 aa, Xenopus laevis
MALWASLALAFASLVAVSQAASLGVVYTEGGFVEGTSKKIGILFPNYIDVFKGIPFAAPPKAFEKAQLHPGWSGTLKATNFKERCLQSTLTQTNVRGDLDCLYLNIWVPQTRSSVSTNLPVMVWIYGGAFLLGSSQGANVLDNYLYDGEELALRGNVIVVTLNYRLGPLGFLSTGDSNLPGNYGLWDQHMAIAWVKRNIAAFGGNPDNITIFGESAGGASVSLQTLSPYNKGLIKRAISQSGVGMSPWALQSNPLFWTTKVAEKVGCPVHDTAAMANCLRISDPKAVTLAYKLDPSVLEYPAVYYLGISPVIDGDFIPDEPMNLFANAADVDYMAGVNNMDAHLFAGIDLPVINQPLQKISEAEVYNLVQGLTLTKISSALETAYNLYTANWGPNPEQENMKRTVIDLETDYLFLVPTQEALALHSMNARSGRTYNYVFSLPTRMPIYPSWVGADHADDLQYVFGKPFQTPLAYRPKDRDVSAAMIAYWTNFAATGDPNQGPSKVPTDWLPYTTHLGQYLEINDNMSYQSVKQSLRSPYVKFWAHTYRNMANV

SEQ ID NO: 22 - DNA, 1662 bp, Xenopus laevis
atggctctttgggcttctcttgccctggcatttgccagtctggtggcagtgtcccaggctgcttcactgggagtggtttacaccgagggtggatttgtggaaggtaccagcaagaaaattgggatcctgttcccgaattacattgatgtttttaagggcatcccgtttgctgctccaccaaaagcctttgagaaggcacaactgcacccaggctggtcaggtacattaaaagccacaaactttaaggaacggtgcttacaatccaccttaacccaaacaaatgtccgtggtgatttagactgcctctacctgaacatctgggttcctcagacccgctcttcagtgtccaccaacctaccagtcatggtttggatctacggtggggccttcttgctcggttcatctcagggggccaacgtgttggataactatctgtatgatggagaggagctcgctctccgtggcaatgtcattgtggtgaccttgaactacagactgggaccattgggctttctgagtactggagactctaaccttcctggcaactatggactgtgggatcaacacatggccatcgcctgggtgaaaaggaacattgctgcatttggtgggaaccctgataacatcaccatatttggagagtctgctggaggcgccagtgtctcccttcagaccctgtctccatacaacaaaggactgatcaagcgagccatcagccagagtggggtgggcatgtccccttgggcacttcagagcaacccacttttctggaccacaaaggtggctgaaaaagttggatgtcctgttcatgacacagccgctatggcaaactgcttgaggatttcagaccctaaggctgtcactttagcctataaactggacccgtctgtcctggagtatcccgctgtttactacttgggcatctccccagtcattgatggtgatttcattcctgatgaaccaatgaatctctttgctaatgcggcggatgtggattacatggcaggtgtaaacaacatggatgcacatttgtttgcaggcatagatctgccagttatcaatcagcctcttcagaagatttctgaggccgaagtctataatctggtgcagggtttgaccctgactaaaatctccagtgccttggaaactgcctacaacctttacacggccaactggggacccaaccctgagcaggagaatatgaaaagaactgtcatagacttagagacggactatcttttcctggtccctacccaagaggcactggctcttcactccatgaatgctcggagtggacggacttacaactacgtgttctctttgccgactcgcatgcccatttaccccagctgggtcggagccgatcatgcagatgatttgcagtacgtgttcgggaaacccttccagactccattggcttacagacccaaggatagagatgtctctgccgccatgattgcctattggaccaactttgctgcaactggtgaccccaaccaaggaccctccaaagtgcccaccgattggctgccttacaccactcaccttggccagtacctggaaatcaacgacaacatgtcttaccaatctgtaaagcagagtctacgttccccttatgtgaaattctgggcccacacttaccgcaatatggccaacgtgtaa

SEQ ID NO: 23 - protein, 556 aa, Gallus gallus
MAHWAILSFALCCCLGVAQAATLGVVLTEGGFVEGESKRRGLFGSYVDIFRGIPFAAPPKALQDPQPHPGWDGTLKAKKFKNRCMQMTLTQTDVRGKEDCLYLNIWIPQGKREVSTNLPVMVWIYGGAFLLGGGQGANFLDNYLYDGEEIAVRGNVIVVTLNYRVGPLGFLSTGDPNMPGNYGLKDQHMAIAWVKRNIKAFGGDPDNITIFGESAGAASVSLQILSPKNAGLFKRAISQSGVSLCSWVIQKDPLTWAKKVGEQVGCPTDNTTVLANCLRATDPKALTLAHHVELISLPGPLVHTLSITPVVDGDFLPDMPENLFANAADIDYIAGVNNMDGHFFAGFDLPAINRPLQKITASDVYNLVKGLTADRGERGANLTYDLYTELWGDNPEQQVMKRTVVDLATDYIFLIPTQWTLNLHHKNARSGKTYSYLFSQPSRMPIYPSWVGADHADDLQYVFGKPFATPLGYLPKHRTVSSAMIAYWTNFARTGDPNSGNSEVPITWPPYTTEGGYYLEINNKINYNSVKQNLRTPYVNYWNSVYLNLPLIASTS

SEQ ID NO: 24 - DNA, 1671 bp, Gallus gallus
atggctcactgggcgattctgagctttgccttgtgctgctgcctcggggtagcacaggccgcaactctgggtgtggtgctcaccgagggaggttttgtggaaggcgagagtaaacgacggggactctttgggagctatgtggatatcttcagagggatcccttttgctgccccgccaaaggcactgcaagacccccaacctcatcctggctgggacggaacactgaaagcaaaaaaatttaagaatcgctgcatgcagatgacacttacccaaactgatgtccgtgggaaggaggactgcctctatctgaacatctggatccctcaagggaagagagaagtctccaccaacttgccagtgatggtctggatctacggtggtgccttccttcttggagggggtcaaggagccaacttccttgacaactacctctatgatggtgaggagatcgccgtgcggggcaatgtgattgtggtgaccctcaactatcgtgtggggcccctgggcttcctcagcactggagacccaaacatgccagggaactacgggctgaaggatcagcacatggctattgcctgggtgaagaggaatatcaaggcctttggaggcgacccagacaacatcaccatctttggggagtcagctggtgctgccagtgtctccctgcagatattgtccccaaagaacgcaggtctgttcaagagagccatcagccaaagcggtgtcagtctgtgcagctgggtcatccaaaaggacccactcacttgggctaaaaaggttggagagcaggtgggctgccccacagacaacaccacggtcttggccaactgcctccgtgccactgaccccaaagccctgacactggcccaccacgtggaactgatctccctgcctggtcccctggttcatacactctccatcactcctgttgttgatggagacttcctccctgacatgccagagaacctctttgccaatgctgctgacatcgactacattgctggggtcaacaacatggatggacatttctttgctggctttgatttacctgctatcaaccgtccacttcagaaaatcactgcgagcgatgtctataacttggtcaaaggactaactgcagacaggggtgagagaggagccaacttgacgtacgatctctacacagagttgtggggtgacaacccagagcaacaagtcatgaagagaacagtggtggacctggctaccgactacattttcctgattcccacacagtggacactaaacctgcaccacaagaatgcccggagtggcaagacatacagctacttgttctcccagccatctcgaatgcccatctatccaagctgggtaggggcagaccacgctgatgacttgcagtacgtgtttgggaaaccctttgccacccctctaggctacctgcccaagcacaggacagtctcatctgccatgattgcttattggaccaattttgccaggactggtgaccccaacagtgggaattcagaggtgcccattacctggccaccctacaccactgagggtggttactacctggaaatcaacaacaaaataaactataattcagtgaaacagaatctgagaaccccatacgtgaactactggaattcagtctatctaaatctgccactgattgccagcacatcctag

SEQ ID NO: 25 - protein, 544 aa, Drosophila melanogaster
MSIFKRLLCLTLLWIAALESEADPLIVEITNGKIRGKDNGLYYSYESIPYAEHPTGALRFEAPQPYSHHWTDVFNATQSPVECMQWNQFINENNKLMGDEDCLTVSIYKPKKPNRSSFPVVVLLHGGAFMFGSGSIYGHDSIMREGTLLVVKISYRLGPLGFASTGDRHLPGNYGLKDQRLALQWIKKNIAHFGGMPDNIVLIGHSAGGASAHLQLLHEDFKHLAKGAISVSGNALDPWVIQQGGRRRAFELGRIVGCGHTNVSAELKDCLKSKPASDIVSAVRSFLVFSYVPFSAFGPVVEPSDAPDAFLTEDPRAVIKSGKFAQVPWAVTYTTEDGGYNAAQLLERNKLTGESWIDLLNDRWFDWAPYLLFYRDAKKTIKDMDDLSFDLRQQYLADRRFSVESYWNVQRMFTDVLFKNSVPSAIDLHRKYGKSPVYSFVYDNPTDSGVGQLLSNRTDVHFGTVHGDDFFLIFNTAAYRIGIRPDEEVISKKFIGMLEDFALNDKGTLTFGECNFQNNVNSKEYQVLRISRNACKNEEYARFP

SEQ ID NO: 26 - DNA, 1635 bp, Drosophila melanogaster
atgagtatattcaaacggctgttgtgcctgactttgctgtggatagcagctttagaatctgaagctgatcccttgattgttgagataacaaatggaaaaatccgtggcaaagataatgggttgtactacagctacgaatcgattccctatgccgagcatccaactggtgccctccgttttgaagcacctcagccgtatagtcatcattggactgatgttttcaatgccacgcagtctccagttgagtgcatgcagtggaatcagtttataaacgaaaacaataagctgatgggtgatgaggattgcttaacggtaagcatctataagccaaagaaacccaatcggagcagctttcctgtcgtagtactcctgcatggaggtgctttcatgttcggtagtggatccatatatggacacgactccattatgcgtgagggaactttgcttgtggtaaaaataagctatcgtcttggaccattgggttttgcaagtaccggcgatagacacttgccgggaaactatggtctaaaggatcaacgtctggccctacaatggatcaagaagaacattgctcactttggtggaatgccagataatattgtgctcattggtcactctgcaggcggtgcttcggctcatttgcagctgttgcacgaggatttcaaacatttggccaaaggagcgatttcggtgagcggcaatgcattggatccttgggtcatacagcagggtggacgacgacgtgcatttgaactgggtcgtattgtcggttgtggacacacaaatgtctccgcagaactcaaggactgcttgaagtctaagccggctagcgatatagtctctgctgtccgaagcttccttgtgttttcctatgtacccttcagtgcttttggacctgttgtggagccgtcagatgcaccagacgcctttctaaccgaggacccaagagcagtgattaagagcgggaagtttgcccaagtcccttgggctgtgacgtacaccactgaggacgggggatacaacgctgctcagctgttggaaagaaacaaattaactggcgagagttggattgacctactcaatgatcgatggtttgattgggcaccatacttgctcttctatcgggacgccaagaaaaccatcaaagatatggatgatctttcatttgatctcaggcagcagtatctagcagatcggcgattcagtgtggaaagttattggaacgtgcagcgaatgtttactgatgttcttttcaagaatagcgtgccaagtgcaatagatcttcaccgaaagtatggcaaaagtccggtttattcttttgtctacgataatcctaccgattccggagtgggtcaattgctttccaatcgaacagatgtacattttggtactgtccacggagatgactttttcttgattttcaatacagctgcataccgtatcggcattcgtccggatgaagaagttatttcaaaaaagtttataggtatgctggaggatttcgcactcaacgataagggaacattaacatttggagaatgtaatttccaaaataatgtgaacagcaaggaatatcaagtgctgcgtatttcacgaaacgcttgtaaaaacgaggaatatgctcggtttccctaa

SEQ ID NO: 27 - protein, 570 aa, Bombyx mori
MCTKFAVLLYYVIVGSVRAYSSPAASPPSSCNVVAQTESGWVCGRTRRAEASTLYASFRGVPYAKQPVGELRFKELQPAEPWTDYLDATEEGPVCYQTDVLYGSLMKPHGMDEACIYANIHVPLNALPAAGETPTKPGLPILVFIHGGGFAFGSGDADLYGPEYLVTRNVVVITFNYRLNFFGFFSLDTPKVPGNNGLRDMVTLLRWVKRNARAFGGNPDNVTLAGQSAGAAAAHLLTLSKATEGLVSRAILMSGAGTSTFFTTSPIFSQSINKILFSILGVNSTNPDEIHEKLVAMPVEKLNEANRILIDQIGLTTFFPVVETPHPGITTILDEDPNILVQQGRGKDIPLIIGFTNSECHMFQHRFEQIDIVSKINENPAILVPSNLLYSSTPETIALVSNQISQRYFNGSVDLEGFINMCTDSYYKYPAMKLAEKRSAAGDAPVFLYQFSYDGYSVFKQAFHLHFNGAGHADDLTYVLKVNSASGTSSSQKADDEMKYWMTTFVTNFMRCSAPMCDETTAWPPVTPRELQYQDIITPNLCHQTSLTKEQLEMKNFFDKIHNGGESRLK

SEQ ID NO: 28 - DNA, 1713 bp, Bombyx mori
atgtgtaccaaattcgctgtattactatattacgttatcgtgggctcagtaagggcatactcaagcccagcagcgtcgccgccgtcgtcgtgcaatgtggtcgcgcagacggagtcaggctgggtgtgtggccgtactcgccgggcggaagcaagcactttatacgccagtttccggggagtgccttatgccaagcaaccagtcggagaacttcgatttaaggaattacaaccagcagagccatggaccgactacctagatgccaccgaggaaggtccagtttgctaccagacagacgttctttatggaagtctaatgaaacctcacggcatggatgaggcatgcatctacgccaatatacatgtgcctttgaacgccctgccggcagctggtgagacgcctacgaagcctggtcttccaatattagtctttattcacggaggtggcttcgcgtttggatctggtgatgctgacctatatggaccggagtatcttgtcacaagaaacgttgttgtcatcacttttaactacaggttgaatttctttggatttttctcattggatactcctaaagtccccggaaacaatggtcttagggacatggtgactctgctccgttgggtgaagaggaacgccagagcctttggaggtaatcctgacaacgtgaccttggcgggccagagcgctggggccgctgctgctcaccttctcaccttatccaaggccactgaaggcttagtttcaagggctatattgatgagcggtgctggaacatccactttctttacaacatctcctatcttctcccagtccatcaacaaaatcttgttttccatccttggcgtcaactccactaatcctgatgagatacacgagaaactcgtcgccatgccggttgaaaaactgaatgaagccaacagaatattgattgatcaaatcggccttaccacttttttcccagtggtagaaacaccgcatcccggaattaccactatattagatgaagatccaaatattctggtccagcaaggccgcggtaaagacatacccttgattataggtttcacgaattctgaatgccatatgttccagcatagatttgaacagatcgatatagtatctaagatcaatgaaaatccagcaatcttagttccttccaatctactgtactcctcgactcctgagacgattgctttggtctcgaatcaaatcagccaaagatacttcaatggtagcgtagatctggagggctttatcaatatgtgtaccgatagttactacaagtacccagccatgaagttggccgagaagagatctgcggcaggtgatgctccggtatttctgtaccagttctcttacgacggttacagcgtgttcaagcaagcctttcatttgcatttcaacggtgctggacacgcggacgacttgacatacgtgctgaaagtgaattctgcgtcagggactagttcatcacaaaaagcagacgatgaaatgaaatattggatgacgacgttcgtcacaaactttatgcgatgcagtgctcctatgtgcgatgaaactacagcgtggccaccagttacaccgcgggaactacaataccaagacattattacaccaaacttatgccaccaaactagtcttaccaaagaacaactcgaaatgaagaatttcttcgataagatccataatggaggtgaaagcagacttaagtaa

SEQ ID NO: 29 - protein, 360 aa, Arabidopsis thaliana
MWTSKTISFTLFITTTLLGSCNASAKAKTQPLFPAILIFGDSTVDTGNNNYPSQTIFRAKHVPYGIDLPNHSPNGRFSNGKIFSDIIATKLNIKQFVPPFLQPNLTDQEIVTGVCFASAGAGYDDQTSLTTQAIRVSEQPNMFKSYIARLKSIVGDKKAMKIINNALVVVSAGPNDFILNYYEVPSWRRMYPSISDYQDFVLSRLNNFVKELYSLGCRKILVGGLPPMGCLPIQMTAQFRNVLRFCLEQENRDSVLYNQKLQKLLPQTQASLTGSKILYSDVYDPMMEMLQNPSKYGFKETTRGCCGTGFLETSFMCNAYSSMCQNRSEFLFFDSIHPSEATYNYIGNVLDTKIRGWLKA

SEQ ID NO: 30 - DNA, 1083 bp, Arabidopsis thaliana
atgtggacttctaaaaccataagcttcactctcttcatcacaacaacacttctcgggtcctgcaacgcatctgcaaaggccaaaacgcaaccgctattcccagcgattctaatctttggtgattcaacagtcgacacaggcaacaataattacccttcacaaacaatcttcagagctaaacatgttccttacggaattgatctcccaaaccactcacctaacggaagattctcaaacgggaaaattttctccgacataatcgcaaccaaactcaacatcaaacagtttgttcctcctttcttacaaccaaatctcaccgaccaagaaattgtaaccggagtctgtttcgcatcagcaggtgccggttacgatgaccaaaccagtctcacgacacaagcgattcgtgtctcggaacaaccaaatatgttcaagagttacattgctcgtcttaagagtatcgtaggagacaagaaagccatgaagatcataaacaatgctttggtggttgtgagtgcagggcctaatgatttcatcttgaattattacgaggttccctcatggcgtcgcatgtatcctagcatttctgattaccaagattttgttcttagtaggcttaacaatttcgtgaaggagctttacagcctaggttgccggaaaattttggtcggaggtttaccgccaatgggatgtttaccgattcaaatgactgctcaattccgcaacgtcctaaggttttgcttggaacaagagaacagagactctgttttatacaatcagaaacttcagaagctcttacctcagacacaagcatctcttacaggaagcaagatcctttactctgatgtctatgaccctatgatggagatgctccaaaaccctagcaaatacggatttaaagagacgacgagaggatgttgtggaacagggttcttggagacgagcttcatgtgtaatgcttattcttccatgtgtcagaatcgctcggagtttctgttctttgactcgattcatccatctgaagctacctacaattacattggtaatgttctggatactaagattcgtgggtggcttaaggcttaa

SEQ ID NO: 31 - protein, 300 aa, Malus pumila
MEPINDEIAREFRFFRVYKDGRIEIFYKTQKVPPSTDEITGVQSKDITIQPEPAVSARIFLPKIHEPAQKLPVLLYLHGGGFIFESAFSPIYHNFVGRLAAEAHAVVVSVEYGLFPDRPVPACYEDSWAALKWLASHASGDGTESWLNKYADFDRLFIGGDSGGANLSHYLAVRVGSLGQPDLKIGGVVLVHPFFGGLEEDDQMFLYMCTENGGLEDRRLRPPPEDFKRLACGKMLIFFAAGDHLRGAGQLYYEDLKKSEWGGSVDVVEHGEGHVFHLFNSDCENAADLVKKFGSFINQK

SEQ ID NO: 32 - DNA, 903 bp, Malus pumila
atggagccaatcaacgacgagattgctcgtgaatttcgcttcttccgggtgtacaaagacggtcgcatagaaatattctacaagacacaaaaggtccccccttcgactgacgaaatcactggtgtccaatccaaggacatcacaattcaacccgaacccgccgtttctgcccgtatcttccttcccaagatccacgagccggcccaaaagctccccgttctcctctacctccacggcggtgggtttatcttcgagtctgccttctctcctatttatcacaacttcgtcggacgattggcagctgaagcccacgcagtcgtagtgtccgtcgaatacgggttgttcccggatcgccccgtacccgcttgctatgaagactcatgggcggcgctcaaatggctcgcgtcccacgctagtggggatggaaccgagtcgtggttaaacaagtatgctgactttgaccggttgtttataggcggggacagcggtggagcaaatttgtcgcactatttggctgtccgggtcgggtccctcgggcaaccggatttgaagattggtggagttgtgctggtgcatccgttctttgggggcttggaggaggacgaccaaatgtttctgtacatgtgtacggagaacggtgggttggaggatcgtaggctgaggccgcccccagaggatttcaaaaggctagcttgcgggaagatgttgatatttttcgcggcgggagaccatctgagaggggcgggccagctgtactatgaggacctgaaaaagagtgagtggggcgggagtgtcgacgtggtggagcatggtgaaggacatgtgtttcacttgttcaattcggactgtgagaatgctgcggacttggtgaaaaaatttggatccttcatcaaccaaaagtag

SEQ ID NO: 33 - DNA, 35 bp, Artificial Sequence
  Primer
cgtctagaaagagaatgatgaaaattgttccgccg

SEQ ID NO: 34 - DNA, 46 bp, Artificial Sequence
  Primer
cggttaacttaatggtgatggtgatggtgccaatctaacgattcaa

SEQ ID NO: 35 - DNA, 762 bp, Artificial Sequence
  CarE-his
atgatgaaaattgttccgccgaagccgtttttctttgaagccggggagcgggcggtgctgcttttgcatgggtttaccggcaattccgccgacgttcggatgcttgggcgattcttggaatcgaaagggtatacgtgccacgctccgatttacaaagggcatggcgtgccgccggaagagctcgtccacaccggaccggatgattggtggcaagacgtcatgaacggctatcagtttttgaaaaacaaaggctacgaaaaaattgccgtggctggattgtcgcttggaggcgtattttctctcaaattaggctacactgtacctacacaaggcattgtgacgatgtgcgcgccgatgtacatcaaaagcgaagaaacgatgtacgaaggtgtgctcgagtatgcgcgcgagtataaaaagcgggaagggaaatcagaggaacaaatcgaacaggaaatggaacggttcaaacaaacgccgatgaagacgttgaaagccttgcaagaactcattgccgatgtgcgcgcccaccttgatttggtttatgcaccgacgttcgtcgtccaagcgcgccatgatgagatgatcaatccagacagcgcgaacatcatttataacgaaattgaatcgccggtcaaacaaatcaaatggtatgagcaatcaggccatgtgattacgcttgatcaagaaaaagatcagctgcatgaagatatttatgcatttcttgaatcgttagattggcaccatcaccatcaccattga

SEQ ID NO: 36 - DNA, 44 bp, Artificial Sequence
  Primer
cgctcgagaaaagagaggctgaagctatgatgaaaattgttccg

SEQ ID NO: 37 - DNA, 29 bp, Artificial Sequence
  Primer
cgcgcgcatattcgaggacgccttcgtac

SEQ ID NO: 38 - DNA, 29 bp, Artificial Sequence
  Primer
cgtcctcgaatatgcgcgcgagtataaaa

SEQ ID NO: 39 - DNA, 47 bp, Artificial Sequence
  Primer
cggaattcttaatggtgatggtgatggtgccaatctaacgattcaag

SEQ ID NO: 40 - DNA, 762 bp, Artificial Sequence
  Mutated CarE-his
atgatgaaaattgttccgccgaagccgtttttctttgaagccggggagcgggcggtgctgcttttgcatgggtttaccggcaattccgccgacgttcggatgcttgggcgattcttggaatcgaaagggtatacgtgccacgctccgatttacaaagggcatggcgtgccgccggaagagctcgtccacaccggaccggatgattggtggcaagacgtcatgaacggctatcagtttttgaaaaacaaaggctacgaaaaaattgccgtggctggattgtcgcttggaggcgtattttctctcaaattaggctacactgtacctacacaaggcattgtgacgatgtgcgcgccgatgtacatcaaaagcgaagaaacgatgtacgaaggcgtcctcgaatatgcgcgcgagtataaaaagcgggaagggaaatcagaggaacaaatcgaacaggaaatggaacggttcaaacaaacgccgatgaagacgttgaaagccttgcaagaactcattgccgatgtgcgcgcccaccttgatttggtttatgcaccgacgttcgtcgtccaagcgcgccatgatgagatgatcaatccagacagcgcgaacatcatttataacgaaattgaatcgccggtcaaacaaatcaaatggtatgagcaatcaggccatgtgattacgcttgatcaagaaaaagatcagctgcatgaagatatttatgcatttcttgaatcgttagattggcaccatcaccatcaccattaa